(19) United States
(12) Patent Application Publication
(10) Pub. No.: US 2024/0173262 A1
(43) Pub. Date: May 30, 2024

(54) MUCOADHESIVE PHARMACEUTICAL DOSAGE FORM FOR UNIDIRECTIONAL RELEASE OF PEPTIDE THERAPEUTIC PARTICLES

(71) Applicant: The University of British Columbia, Vancouver (CA)

(72) Inventors: Anubhav Pratap-Singh, Vancouver (CA); Yigong Guo, Vancouver (CA); Anika Singh, Vancouver (CA); Farahnaz Fathordoobady, Vancouver (CA); David D. Kitts, Vancouver (CA); Alberto Baldelli, Vancouver (CA)

(73) Assignee: The University of British Columbia, Vancouver (CA)

(21) Appl. No.: 18/513,373

(22) Filed: Nov. 17, 2023

Related U.S. Application Data
(63) Continuation of application No. PCT/CA2023/051129, filed on Aug. 25, 2023.
(60) Provisional application No. 63/400,863, filed on Aug. 25, 2022.

(51) Int. Cl. A61K 9/20; A61K 9/00; A61K 38/28; A61P 3/10

(52) U.S. Cl. CPC A61K 9/2086; A61K 9/006; A61K 9/2027; A61K 9/2054; A61K 9/2081; A61K 38/28; A61P 3/10

(57) ABSTRACT

Provided herein are pharmaceutical dosage forms for delivering peptide therapeutics to a mucosal surface. The pharmaceutical dosage forms comprise a mucoadhesive layer; a peptide loading layer; and a water impermeable layer, wherein the peptide loading layer is between the mucoadhesive layer and the water impermeable layer, such that the water impermeable layer facilitates unidirectional movement of the encapsulated peptide through the mucoadhesive layer and to the target mucosal surface. The pharmaceutical dosage form is suitable for delivery of the encapsulated peptide therapeutic to buccal mucosa; sublingual mucosa; palate mucosa; and tongue mucosa. The peptide therapeutic may be an insulin; an insulin derivative; an insulin analog; a pre-insulin; a pro-drug of insulin; a glucagon-like peptide 1 (GLP-1); a GLP-1 analog; a pre- GLP-1; a pro-drug of GLP-1; or a combination thereof and may be suitable for the treatment of diabetes.

### CROSS REFERENCE TO RELATED APPLICATIONS

This application is a continuation of International Application No. PCT/CA2023/051129 filed Aug. 25, 2023, which claims the benefit of U.S. Provisional Patent Application Ser. No. 63/400,863 filed 25 Aug. 2022, the disclosure of each of which is expressly incorporated herein by reference in its entirety.

### TECHNICAL FIELD

The present invention relates to pharmaceutical dosage forms for delivery of encapsulated peptides to the oral mucosa and methods and uses for the pharmaceutical dosage forms. In particular, herein are provided pharmaceutical dosage forms having a mucoadhesive layer, a peptide loading layer and a water impermeable layer. The peptide loading layer includes an encapsulated peptide. In particular, the encapsulated peptide may be a peptide particle coated with chitosan. Furthermore, the encapsulated peptide may be selected from: an insulin; an insulin derivative; an insulin analog; a pre-insulin; a pro-drug of insulin; a glucagon-like peptide 1 (GLP-1); a GLP-1 analog; a pre- GLP-1; a pro-drug of GLP-1; or a combination thereof. The pharmaceutical dosage forms may be used for the treatment of diabetes or for the treatment of obesity. The pharmaceutical dosage forms may be used to deliver the encapsulated peptide to a subject's oral mucosa. In particular, the oral mucosa may be selected from one or more of: buccal mucosa; sublingual mucosa; palate mucosa; and tongue mucosa.

### BACKGROUND

Injectable peptides like insulin and glucagon-like peptide 1 (GLP-1) and their analogs are increasingly being used for treatment of diabetes and obesity. Insulin is a mainstay of treatment for insulin-dependent type 1 diabetes¹. It is also used for the treatment of non-insulin-dependent type 2 diabetes². Due to insulin's tendency to be used as a long-term treatment, its delivery via injections has several disadvantages concerning patient acceptance. For instance, patients with non-insulin dependence often delay the initiation of their insulin therapy. The perception of insulin injections or needle anxiety is one of the factors associated with this delayed start phenomenon, which has been called psychological insulin resistance³. As a result of these parenteral concerns, many attempts have been made to develop alternative delivery routes, resulting in mixed results as reported in the literature. In particular, the oral route has been extensively explored⁴-^{6, 42, 43}. For oral delivery of peptide formulations, there are numerous challenges, such as low gastric pH, proteolysis enzymes in the upper gastrointestinal tract, and low absorption and low bioavailability. Although the encapsulation of insulin can overcome some of these drawbacks, traditional oral delivery followed by gastrointestinal tract adsorption of encapsulated insulin is considered unpredictable due to varying absorption and slow onset of action compared with an injection⁷. Therefore, finding an alternative route that can have the same fast onset of action as an injection while being administrated more conveniently would be of interest.

Alternative routes of administration have been investigated to circumvent the disadvantages of the gastrointestinal tract, such as pulmonary, nasal, transdermal, and buccal administration methods^{8, 9}. Among the various methods for delivering insulin, mucosal administration has the advantage of a non-keratinized epithelium which might result in improved absorption compared to the characteristics of a keratinized epithelium. In particular, delivery via the oral mucosa has several advantages, including a relatively low enzyme content compared to the gastrointestinal tract, good drainage of the vascular and lymphatic systems, potential ease in administration, and a long cellular turnover (5-6 days) that may enable the delivery of the drug in retentive dosage forms for an extended period of time¹⁰. There are several reasons, however, why drugs are challenging to deliver via the oral mucosa, including the limited absorption area, the barrier properties and the involuntary swallowing of a tablet, and continuous dilution of the dissolved drug by saliva.

Thin films have been used to create a larger absorption area and mucoadhesive thin films have been used to avoid swallowing of the dosage form^{12, 41, 46}.

The application of peptide encapsulation in delivering insulin has also been explored. The idea of providing insulin particles through buccal tissue has been published in a few research studies^{8, 11}. Chitosans have been used in mucoadhesive tablets and films for drug delivery⁴⁴. Furthermore, thiolated chitosans have been explored for improved mucoadhesion⁴⁵. However, the methods mostly focused on buccal films or patches and lacked in vivo data^{12, 13}.

### SUMMARY

The present invention is based, in part, on the surprising discovery that a particular mucoadhesive layers are particularly useful in creating pharmaceutical dosage forms suitable for delivery of peptides via the oral mucosa. In particular, the present mucoadhesive layer is able to minimize unwanted loss of peptides to saliva due to early release of peptides, while mucoadhesive layer is still forming attachments to the oral mucosa. Furthermore, having the peptide in the mucoadhesive layer might also result in lost mucoadhesivity, whereby having peptide in peptide loading layer complexed with chitosan improves bioavailability. Furthermore, including the peptides in the mucoadhesive layer also causes very rapid peptide release that may result in loss of peptide to saliva, where peptides are not absorbed if the cell tight junctions are not yet open. Or where the tight junctions are open, the presence of the peptides in the mucoadhesive layer would not have a prolonged release.

Furthermore, the present invention is also based, in part, on the surprising discovery that “encapsulated peptides” having a peptide particle coated with a mercaptonicotinic acid (MNA)-thioglycolytic acid-chitosan (MNA-TG-chitosan) demonstrated a higher cell uptake than free peptide or other encapsulated peptides. MNA-TG-chitosan particles showed the highest mucosal penetration within 2 hours, followed by peptide-loaded TG-chitosan particles and peptide-loaded chitosan particles.

The new coating material (i.e., MNA-TG-chitosan) was found to be suitable for oral mucosa delivery of encapsulated insulin in uni-directional oral mucosa tablets. Compared with the existing buccal film, the three layer oral mucosa tablets were prepared to achieve uni-directional drug release toward the oral mucosa.

One strategy that may overcome these obstacles is the application of mucoadhesive buccal tablets made of polymers. Mucoadhesive tablets are useful for oral mucosal delivery of peptides due to their small size and user-friendly properties, which facilitates patient compliance.

Furthermore, it was fortuitously discovered that having a mucoadhesive layer of at least 1 micrometer is useful to obtain the peptide release characteristics suitable for insulin GLP-1 etc. Furthermore, having a mucoadhesive layer that is between 100 μm to 1,500 μm is preferred for most peptide release dosage forms.

In a first embodiment, there is provided a pharmaceutical dosage form, the pharmaceutical dosage form may include: (a) a mucoadhesive layer, the mucoadhesive layer may include: (i) an ethylcellulose and a polymer of acrylic acid cross-linked with allyl sucrose or allyl pentaerythritol, wherein the polymer of acrylic acid cross-linked with allyl sucrose or allyl pentaerythritol is at least 12.5 wt % of the total mucoadhesive layer; or (ii) an ethylcellulose, a polymer of acrylic acid cross-linked with allyl sucrose or allyl pentaerythritol, wherein the polymer of acrylic acid cross-linked with allyl sucrose or allyl pentaerythritol is at least 12.5 wt % of the total mucoadhesive layer, and a hydroxypropyl methylcellulose (HPMC); (b) a peptide loading layer, the peptide loading layer may include an encapsulated peptide; and (c) a water impermeable layer, selected from one or more of ethylcellulose; polyvinylchloride; polydimethylsiloxane; hydroxypropyl methylcellulose; hemicellulose; Poly(e-caprolactone) (PCL); carboxymethyl cellulose; polyvinylacetate; propylcellulose; polymethyl methacrylate; methacrylic acid copolymer; and cellulose acetate phthalate; wherein the peptide loading layer resides between the mucoadhesive layer and the water impermeable layer, and wherein the water impermeable layer facilitates unidirectional movement of the encapsulated peptide through the mucoadhesive layer to a target tissue.

In a further embodiment, there is provided a pharmaceutical dosage form, the pharmaceutical dosage form may include: (a) a mucoadhesive layer, the mucoadhesive layer may include one of (i) or (ii) or (iii): (i) an ethylcellulose and a polymer of acrylic acid cross-linked with allyl sucrose or allyl pentaerythritol, wherein the polymer of acrylic acid cross-linked with allyl sucrose or allyl pentaerythritol is at least 12.5 wt % of the total mucoadhesive layer; or (ii) an ethylcellulose, a polymer of acrylic acid cross-linked with allyl sucrose or allyl pentaerythritol, wherein the polymer of acrylic acid cross-linked with allyl sucrose or allyl pentaerythritol is at least 12.5 wt % of the total mucoadhesive layer, and a hydroxypropyl methylcellulose (HPMC); or (iii) an ethylcellulose and a polyvinylpyrrolidone (PVP); (b) a peptide loading layer, the peptide loading layer may include an encapsulated peptide; and (c) a water impermeable layer, selected from one or more of ethylcellulose; polyvinylchloride; polydimethylsiloxane; hydroxypropyl methylcellulose; hemicellulose; Poly(e-caprolactone) (PCL); carboxymethyl cellulose; polyvinylacetate; propylcellulose; polymethyl methacrylate; methacrylic acid copolymer; and cellulose acetate phthalate; wherein the peptide loading layer resides between the mucoadhesive layer and the water impermeable layer, and wherein the water impermeable layer facilitates unidirectional movement of the encapsulated peptide through the mucoadhesive layer to a target tissue.

The water impermeable layer may form an outer coating while leaving a mucoadhesive surface without the water impermeable layer, to facilitate muco-adhesion. Alternatively, the water impermeable layer may form an outer coating where at least a portion of the mucoadhesive surface is not covered by the water impermeable layer, to facilitate muco-adhesion. The ethylcellulose in (i) or (ii) or (iii) may be: at least 50 wt % of the total mucoadhesive layer; or may be between about 50 wt % and about 75 wt % of the total mucoadhesive layer. The ethylcellulose in (i) or (ii) or (iii) may be: at least 40 wt % of the total mucoadhesive layer; or may be between about 40 wt % and about 80 wt % of the total mucoadhesive layer. The ethylcellulose in (i) or (ii) or (iii) may be: at least 50 wt % of the total mucoadhesive layer; or may be between about 50 wt % and about 87.5 wt % of the total mucoadhesive layer. The polymer of acrylic acid cross-linked with allyl sucrose or allyl pentaerythritol may be between about 12.5 wt % and about 50 wt % of the total mucoadhesive layer. The polymer of acrylic acid cross-linked with allyl sucrose or allyl pentaerythritol may be between about 10 wt % and about 50 wt % of the total mucoadhesive layer. The polymer of acrylic acid cross-linked with allyl sucrose or allyl pentaerythritol may be between about 10 wt % and about 40 wt % of the total mucoadhesive layer. The polymer of acrylic acid cross-linked with allyl sucrose or allyl pentaerythritol may be between about 12.5 wt % and about 40 wt % of the total mucoadhesive layer. The polymers of acrylic acid cross-linked with allyl sucrose or allyl pentaerythritol may be selected from one or more of the following: Carbopol 934 NF™; Carbopol 934P NF™; Carbopol 941 NF™; Carbopol 942NF™; Carbopol 940NF™; Carbopol 974P™; Carbopol 971P™; Noveon AA-1 Polycarbophil™ (formerly Carbopol 976™); Carbopol 1342™; Carbopol 1382™; Carbopol salts™; Carbopol 981 NF™; Carbopol 980 NF™; Carbopol ETD 2050™; Carbopol ETD 2020™; Carbopol ULTREZ 10™; Carbopol 1984™; Carbopol 2984™; Carbopol 5984™; and Carbopol(R) 71G NF™. The polymers of acrylic acid cross-linked with allyl sucrose or allyl pentaerythritol may be selected from one or more of the following: Carbopol 934 NF™; Carbopol 934P NF™; Carbopol 941 NF™; Carbopol 942NF™; and Carbopol 940NF™. The polymers of acrylic acid cross-linked with allyl sucrose or allyl pentaerythritol may be Carbopol 934P NF™.

The mucoadhesive layer may have a thickness of at least 100 μm. The mucoadhesive layer may have a thickness of at least 50 μm. Alternatively, the mucoadhesive layer may have a thickness of at least 1 micrometer. Preferably, the mucoadhesive layer may have a thickness between 100 μm to 1,500 μm. The mucoadhesive layer may have a thickness between 100 μm to 1,000 μm. The mucoadhesive layer may have a thickness between 100 μm to 2,000 μm. The mucoadhesive layer may have a thickness between 100 μm to 900 μm. The mucoadhesive layer may have a thickness between 100 μm to 800 μm. The mucoadhesive layer may have a thickness between 100 μm to 700 μm. The mucoadhesive layer may have a thickness between 100 μm to 600 μm. The mucoadhesive layer may have a thickness between 100 μm to 500 μm. The mucoadhesive layer may have a thickness between 100 μm to 400 μm. The mucoadhesive layer may have a thickness between 100 μm to 300 μm. The mucoadhesive layer may have a thickness between 100 μm to 200 μm.

The encapsulated peptide may include a peptide particle coated with chitosan. The encapsulated peptide my include a tripolyphosphate (TPP), a chitosan, and a peptide therapeutic. The chitosan may be a thiolated chitosan. The thiolated chitosan may be selected from: MNA-TG-chitosan; and TG-chitosan. The encapsulated peptide may be a particle having a diameter between about 50 nm and about 10,000 nm. The encapsulated peptide may be a particle having a diameter between about 50 nm and about 9,000 nm. The encapsulated peptide may be a particle having a diameter between about 50 nm and about 8,000 nm. The encapsulated peptide may be a particle having a diameter between about 50 nm and about 7,000 nm. The encapsulated peptide may be a particle having a diameter between about 50 nm and about 6,000 nm. The encapsulated peptide may be a particle having a diameter between about 50 nm and about 5,000 nm. The encapsulated peptide may be a particle having a diameter between about 50 nm and about 4,000 nm. The encapsulated peptide may be a particle having a diameter between about 50 nm and about 3,000 nm. The encapsulated peptide may be a particle having a diameter between about 50 nm and about 2,000 nm. The encapsulated peptide may be a particle having a diameter between about 50 nm and about 1,000 nm. The encapsulated peptide may be a particle having a diameter between about 50 nm and about 900 nm. The encapsulated peptide may be a particle having a diameter between about 50 nm and about 800 nm. The encapsulated peptide may be a particle having a diameter between about 50 nm and about 700 nm. The encapsulated peptide may be a particle having a diameter between about 50 nm and about 6900 nm. The encapsulated peptide may be a particle having a diameter between about 50 nm and about 500 nm. The encapsulated peptide may be a particle having a diameter between about 50 nm and about 400 nm. The encapsulated peptide may be a particle having a diameter between about 100 nm and about 400 nm. The encapsulated peptide may be a particle having a diameter between about 150 nm and about 400 nm.

The encapsulated peptide may limited to the peptide loading layer prior to administration. The encapsulated peptide may elute through the mucoadhesive layer following muco-adhesive attachment to an oral mucosa.

The encapsulated peptide may be synthesized by 1-Ethyl-3-(3-dimethylaminopropyl)carbodiimide (EDC) coupling of chitosan and thioglycolytic acid (TGA), followed by thiol-disulfide exchange of the thiolated chitosan with 2,2′-disulfandiylnicotinic acid.

The peptide loading layer may further include: one or more sweetener or flavourant. The one or more sweetener or flavourant may be selected from one or more of: lactose; glucose; sucrose; mannitol; xylitol; sorbitol; and trehalose. The sweetener may be mannitol. The peptide loading layer may further include a binding agent. The binding agent may be sodium alginate.

The encapsulated peptide may be selected from an insulin; an insulin derivative; an insulin analog; a pre-insulin; a pro-drug of insulin; a glucagon-like peptide 1 (GLP-1); a GLP-1 analog; a pre- GLP-1; a pro-drug of GLP-1; or a combination thereof. The insulin analog may be selected from one or more of: lispro; aspart; glulisine; glargine; detemirt; detemir; degludec; neutral protamine hagedorn (NPH); and levemir. The GLP-1 may be selected from one or more of: exenatide; liraglutide; dulaglutide; and semaglutide.

The target tissue may be an oral mucosa. The oral mucosa may be selected from one or more of: buccal mucosa; sublingual mucosa; palate mucosa; and tongue mucosa.

The mucoadhesive layer may have a mucoadhesivity between about 1 N and about 30 N. The mucoadhesive layer may have a mucoadhesivity between about 1 N and about 25 N. The mucoadhesive layer may have a mucoadhesivity between about 1 N and about 20 N. The mucoadhesive layer may have a mucoadhesivity between about 1 N and about 15 N. The mucoadhesive layer may have a mucoadhesivity between about 1 N and about 10 N. The mucoadhesive layer may have a mucoadhesivity between about 1 N and about 9 N. The mucoadhesive layer may have a mucoadhesivity between about 1 N and about 8 N. The mucoadhesive layer may have a mucoadhesivity between about 1 N and about 7 N. The mucoadhesive layer may have a mucoadhesivity between about 1 N and about 6 N. The mucoadhesive layer may have a mucoadhesivity between about 1 N and about 5 N. The mucoadhesive layer may have a mucoadhesivity between about 1 N and about 4 N. The mucoadhesive layer may have a mucoadhesivity between about 1 N and about 3 N. The mucoadhesive layer may have a mucoadhesivity between about 1 N and about 2 N.

The pharmaceutical dosage form may be a tablet dosage form.

The pharmaceutical dosage form may be for the treatment of diabetes. The pharmaceutical dosage form may be for the treatment of obesity. The pharmaceutical dosage form may be for the treatment of diabetes and obesity.

In a further embodiment, there is provided a use of the pharmaceutical dosage form as described herein for the treatment of diabetes.

In a further embodiment, there is provided a use of the pharmaceutical dosage form as described herein, in the manufacture of a medicament for the treatment of diabetes.

In a further embodiment, there is provided a use of the pharmaceutical dosage form as described herein for the treatment of obesity. In a further embodiment, there is provided a use of the pharmaceutical dosage form as described herein, in the manufacture of a medicament for the treatment of obesity.

The diabetes may be insulin-dependent diabetes type 1 (T1D) or type-2 diabetes (T2D).

In a further embodiment, there is provided a method of treating diabetes, including administering the pharmaceutical dosage form described herein, to a subject in need thereof.

In a further embodiment, there is provided a method of treating obesity, including administering the pharmaceutical dosage form described herein, to a subject in need thereof.

In a further embodiment, there is provided a use of the pharmaceutical dosage form described herein, for oral mucosa delivery of the encapsulated peptide.

In a further embodiment, there is provided a method for oral mucosa delivery of a peptide, comprising administering the pharmaceutical dosage form described herein to a subject in need thereof.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows the characterization of TG-chitosan and preactivated MNA-TG-chitosan synthesis, wherein (A) shows ATR-FTIR of unmodified chitosan, TG-chitosan and MNA-TG-chitosan and (B) shows ¹H NMR-characterization of preactivated MNA-TG-chitosan.

FIG. 2 shows the characterization of insulin particles coated with different chitosans, wherein (A) shows the size distribution of insulin particles coated with chitosan, TG-chitosan, and MNA-TG-chitosan after reconstitution; (B) shows TEM micrographs of the optimized insulin particles; (C) shows SEM image of spray dried insulin coated with chitosan, TG-chitosan, and MNA-TG-chitosan; and (D) shows ATR-FTIR spectra for free insulin, chitosan, a physical mixture of chitosan/TPP/insulin and insulin particles dehydrated by different methods.

FIG. 3 shows the swelling index of (A) Buccal tablets with pure insulin; (B) Buccal tablets with insulin particles coated with chitosan; (C) Buccal tablets with insulin particles coated with TG-chitosan; and (D) Buccal tablets with insulin particles coated with MNA-TG-chitosan.

FIG. 4 shows penetration behavior of (A) Buccal tablets with pure insulin; (B) Buccal tablets with insulin particles coated with chitosan; (C) Buccal tablets with insulin particles coated with TG-chitosan; (D) Buccal tablets with insulin particles coated with MNA-TG-chitosan; and (E) Back layers of prepared tablets.

FIG. 5 shows HepG2 cellular uptakes after 4 h incubation with free insulin and insulin particles: (A) Distribution of FITC-insulin uptaken by HepG2 cells; and (B) Geometric mean values of the fluorescence intensities of the flow cytometry analysis.

FIG. 6 shows (A) the effects of insulin particles coated with chitosan on TEER values of TR-146 cell monolayer; (B) fluorescence images of TR-146 monolayer stained for tight junction protein ZO-1 after incubation with insulin particles; (C) the effect of particles on transepithelial transport across TR-146 monolayer; and (D) Pₐₚₚ values of insulin particles across the TR-146 cell monolayer.

FIG. 7 shows oral delivery of insulin particles and buccal tablets on blood glucose level in a diabetic rat model, wherein (A) Blood glucose level vs. time profiles of diabetic rats; and (B) serum insulin level vs. time profiles of normal rats.

FIG. 8 shows the biodistribution of orally delivered insulin particles and insulin buccal tablets as compared to i.p. injection of insulin.

FIG. 9 shows enzyme activity in serum (U/L) as detected to determine whether administration of insulin buccal tablets was safe (alkaline phosphatase (ALP), aspartate transaminase (AST) and alanine aminotransferase (ALT)).

FIG. 10 shows mucoadhesion strength measurement apparatus (a) TA.XTplus texture analyzer set up; and (b) strength measurement result recorded from the mucoadhesion strength measurement apparatus.

FIG. 11 shows an ex-vivo penetration experimental set up to test tablet dosage forms on porcine mucosal tissues.

FIG. 12 shows graphic representations of mucoadhesive tablets (a) and films (b) for the oral mucosa delivery of peptides.

### DETAILED DESCRIPTION

The following detailed description will be better understood when read in conjunction with the appended figures. For the purpose of illustrating the invention, the figures demonstrate embodiments of the present invention. However, the invention is not limited to the precise arrangements, examples, and instrumentalities shown. Any terms not directly defined herein shall be understood to have the meanings commonly associated with them as understood within the art of the invention.

### Definitions

As used herein “polymers of acrylic acid cross-linked with allyl sucrose or allyl pentaerythritol” are also known as carboxypolymethylene, polyacrylic acid or poly(1-carboxyethylene), carbomers, carbopol, or the acrylic acid may be represented as (C₃H₄O₂)ₙ or as shown below, where n=3×10⁶ for Carbomer 934; 4×10⁶ Carbomer 940; or 1×10⁶ for Carbomer 941. Examples, include but are not limited to Carbopol 934 NF™; Carbopol 934P NF™; Carbopol 941 NF™; Carbopol 942NF™; Carbopol 940NF™; Carbopol 974P™; Carbopol 971P™; Noveon AA-1 Polycarbophil™(formerly Carbopol 976™); Carbopol 1342™; Carbopol 1382™; Carbopol salts™; Carbopol 981 NF™; Carbopol 980 NF™; Carbopol ETD 2050™; Carbopol ETD 2020™; Carbopol ULTREZ 10™; Carbopol 1984™; Carbopol 2984™; Carbopol 5984™; and Carbopol(R) 71G NF™

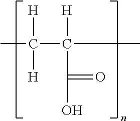

As described herein there are provided encapsulated peptide formulations, methods for the preparation of encapsulated peptides using said formulations, and particle compositions for the delivery of encapsulated peptides to the desired site of action.

As used herein, polyvinylpyrrolidone (PVP) is a polymer that may be used in pharmaceutical dosage forms described herein. In particular, PVPs may be used as an alternative to the carbomers in the mucoadhesive layer of the tablet dosage form, due to the favourable solubility and mucoadhesivity of these polymers.

As used herein, “sweeteners” may refer to sugars or sugar alcohols. For example, D-Mannitol ((CAS No.: 69-65-8) M4125, Sigma Aldrich™); Lactose (61345, Sigma Aldrich™); and Trehalose (T9449 D-(+)-Trehalose dihydrate, Sigma Aldrich™). Also, the sweeteners may be used alone or in combination as described herein. As used herein sugar means soluble carbohydrates such as monosaccharides, disaccharides and polysaccharides and commonly exemplified by glucose and sucrose. As described herein the sugar is preferably lactose. As used herein sugar alcohol (also called polyhydric alcohols, polyalcohols, alditols or glycitols) refers to organic compounds derived from sugars, containing one hydroxyl group (—OH) attached to each carbon atom. Sugar alcohols are often used as artificial sweeteners and is exemplified by xylitol and sorbitol. As described herein, the sugar alcohol is preferably mannitol.

As used herein “encapsulated peptides” or “encapsulated peptide particles” are meant to include particles having a diameter between about 50 nm and about 10,000 nm. More preferably, the particles may have a diameter between about 150 and about 400 nm.

As used herein an “encapsulated peptide” includes a peptide particle coated with chitosan or a thiolated chitosan, whereby the thiol groups (—SH) groups on thiolated chitosan may form disulphide or other linkages. The thiolated chitosan may be selected from: mercaptonicotinic acid (MNA)-thioglycolytic acid-chitosan (MNA-TG-chitosan); and thioglycolytic acid-chitosan (TG-chitosan), or other chitosans known to a person of skill^{47, 48}.

Peptide particles as described herein may be prepared by spray freeze drying, spray drying, or freeze drying.

As used herein, “spray freeze drying” means the process of drying a material involving a solution being atomized, solidified and sublimed at low temperature. The atomized material is typically solidified by rapid freezing into a cryogenic fluid such as liquid nitrogen (LN2) along with additional excipients that protect the structure, activity and stability of said material. The mixture then undergoes further drying by freeze-drying in a vacuum chamber to remove residual moisture. As described herein, formulations for preparation of proteins by spray freeze drying, which are normally sensitive to degradation by heat, moisture or chemical/enzymatic action are provided. This is of particular concern when preparing therapeutic proteins that requires its structure and/or biological activity to be preserved until it is delivered to the desired site of action.

As used herein, “spray drying” means the process of forming a dry powder from a liquid or slurry by rapidly drying with a hot gas. The process usually takes a solution or suspension to be dried and atomizes the solution or suspension with an atomization gas. The atomized solution or suspension is sprayed into a drying gas, where the drying gas is heated. The sprayed solution or suspension dries in the heated drying gas to produce fine particles.

As used herein “freeze drying” or “lyophilization” is a method of low temperature dehydration. Freeze drying involves freezing the peptide solution at low pressure to remove the ice by sublimation, which is in contrast to dehydration by most conventional methods that evaporate water using heat which could denature a peptide or protein.

An “effective amount” of an active ingredient as described herein includes a therapeutically effective amount or a prophylactically effective amount. A “therapeutically effective amount” refers to an amount effective, at dosages and for periods of time as needed, to achieve the desired therapeutic result, such as prolonged peptide delivery, glucose homeostasis, increased life span or increased life expectancy. A therapeutically effective amount of an active ingredient may vary according to factors such as the disease state, age, sex, and weight of the subject, and the ability of the active ingredient to elicit a desired response in the subject. Dosage regimens may be adjusted to provide the optimum therapeutic response. A therapeutically effective amount is also one in which any toxic or detrimental effects of the active ingredient are outweighed by the therapeutically beneficial effects. A “prophylactically effective amount” refers to an amount effective, at dosages and for periods of time necessary, to achieve the desired prophylactic result, such as prolonged peptide delivery, glucose homeostasis, increased life span, increased life expectancy or prevention of the diabetes or obesity. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active ingredient(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage. As specifically described herein most of the particle compositions are tailored for oral mucosa delivery.

An encapsulated peptide, as described herein, may be administered to a subject. As used herein, a “subject” may be a human, non-human primate, rat, mouse, cow, horse, pig, sheep, goat, dog, cat, etc. The subject may be suspected of having or at risk for having diabetes, such as Type 1 Diabetes (T1D) or Type 2 diabetes (T2D). Alternatively, the subject may be suspected of having or at risk for having obesity.

Any terms not directly defined herein shall be understood to have the meanings commonly associated with them as understood within the art.

Various alternative embodiments and examples are described herein. These embodiments and examples are illustrative and should not be construed as limiting the scope of the invention.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Various alternative embodiments and examples are described herein. These embodiments and examples are illustrative and should not be construed as limiting the scope of the invention.

### Materials and Methods

### Materials

Chitosan (Average Mw 100 KDa, 75-85% deacetylated), Carbopol 934P™, hydroxypropyl methylcellulose, and ethylcellulose were purchased from Sigma-Aldrich™ (Oakville, Ontario, Canada). Sodium tripolyphosphate (TPP) and sodium alginate (low viscosity) was purchased from VWR™ (Radnor, Pennsylvania, USA). Recombinant human insulin used in this study was from Fisher Scientific™ (Waltham, Massachusetts, USA). Fluorescein isothiocyanate (FITC)-labeled human insulin and the 4′, 6-Diamidino-2-phenylindole dihydrochloride (DAPI) were purchased from Sigma-Aldrich™ (Oakville, Ontario, Canada). The HepG2 and Caco-2 cell line was obtained from ATCC (Manassas, Virginia, USA) while TR-146 Cell line was purchased from Sigma-Aldrich™ (Oakville, Ontario, Canada). All the other reagents were of analytical or chromatography grade.

### Synthesis and Purification of MNA-TG-chitosan

### Synthesis and Purification of Thiolated Chitosan

Thiolated chitosan (TG-chitosan) was synthesized using a method previously described¹⁴. The synthesis route is shown below in Synthesis 1. Chitosan was first dissolved in 0.1M of HCl. Then, thioglycolic acid (TGA) was added and the pH was adjusted to 5.0 with NaOH. 50 mM of EDAC was added to activate the carboxylic moiety of TGA. The resultant thiolated chitosan was dialyzed to remove unconjugated TGA.

### Synthesis and Purification of Preactivated TG-chitosan (MNA-TG-Chitosan)

TG-chitosan was subsequently utilized to synthesize mercaptonicotinic acid preactivated TG-chitosan (MNA-TG-chitosan). The synthesis route was shown in below in Synthesis 1. Preactivation was carried out by adding drops of MNA solution to TG-chitosan solution with a ratio of 1:2. The pH of the reaction mixture was set to 7.5 and stirred continuously for six hours at room temperature. Following the reaction, the MNA-TG-chitosan was purified using a dialysis procedure, as well. Purified MNA-TG-chitosan were then frozen, dried, and stored at 4° C. in the dark until further use.

### Synthesis 1: Synthesis Routes for TG-chitosan and MNA-TG-chitosan.

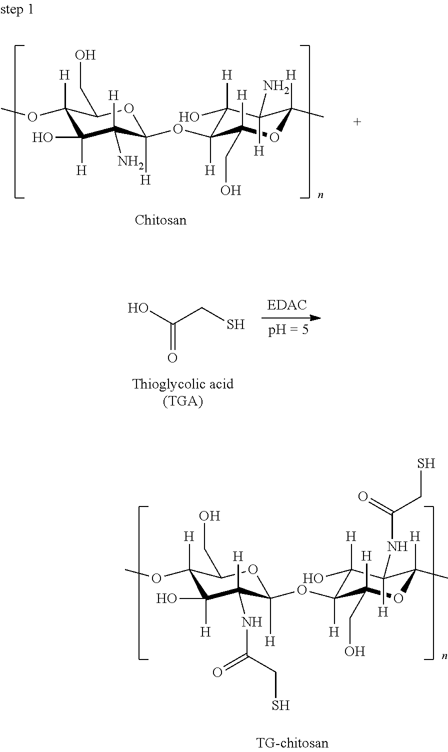

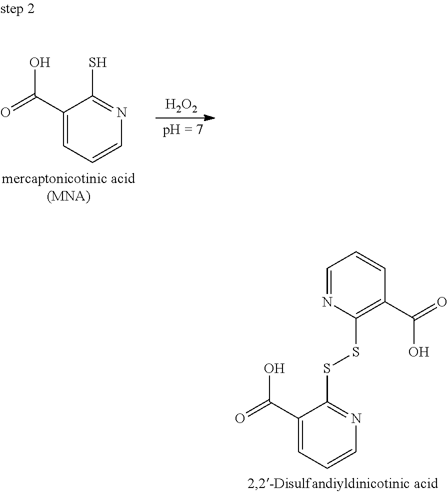

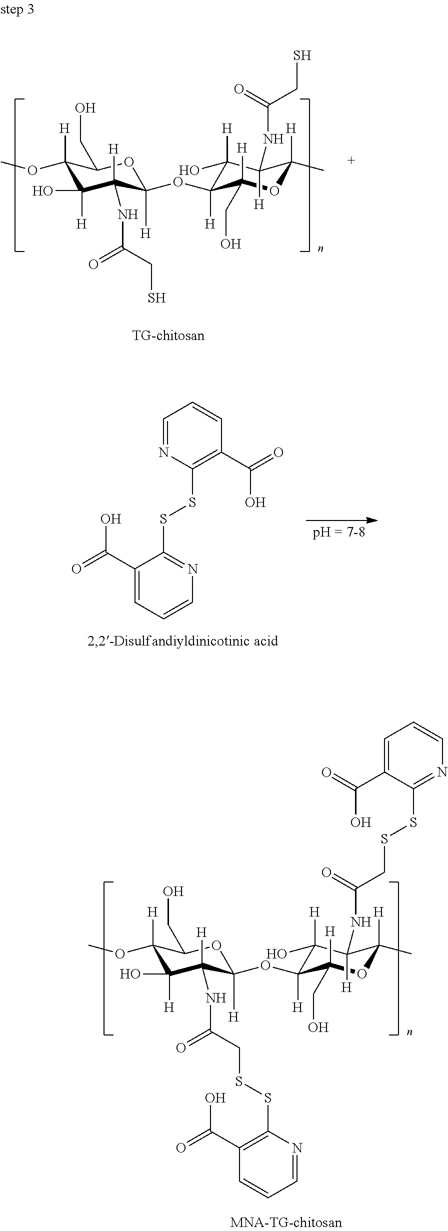

### Characterization of TG-chitosan and MNA-TG-Chitosan via Ellman's Assay, FT-IR and NMR

As described previously³⁶, Ellman's assay determined the concentration of thiol groups and disulfide bonds in freeze-dried MNA-TG-chitosan and TG-chitosan. For thiol group determination, briefly, TG-chitosan was mixed with phosphate buffer and incubated at room temperature for 30 min. Then, Ellman's reagent was added and incubated at room temperature for 90 min in the dark. After 90 min, aliquots of 100 μL were transferred to a 96-well plate and the absorbance was measured at 450 nm by Tecan Infinite M200™ pro spectrophotometer plate reader (Tecan™, Mannedorf, Switzerland). For disulfide bonds determination, MNA-TG-chitosan was dissolved in 50 mM Tris buffer. Then, 4% sodium borohydride solution was added and incubated at 370C for 60 min. After the incubation, HCl was added, followed by Ellman's reagent and further incubated for 90 min at room temperature in the dark.

The FTIR of TG-chitosan and MNA-TG-chitosan were conducted by Spectrum 100™ FTIR spectrophotometer (PerkinElmer™, Waltham, Massachusetts, USA) equipped with universal ATR sampling accessories (PerkinElmer™, Waltham, Massachusetts, USA). Signal averages were obtained from 16 scans in the frequency range of 4000-600 cm² at a resolution of 4 cm².

Furthermore, ¹H-NMR spectra were taken on a Bruker Avance Cryoprobe™ 600 MHz spectrometer (Bruker™, Billerica, Massachusetts, USA). The data was acquired and processed with MestReNova Ver. 12.0.4™ (Mestrelab Research™, Santiago de Compostela, Spain). ¹H NMR spectra were got by Bruker™ pulse programs with standard acquisition parameters at 298K (25° C.).

### Insulin Particles

### Insulin Particle Preparation

Insulin particles were prepared using the optimized method from our previous research¹⁰. Briefly, the cross linked TPP/insulin solution was mixed at a ratio of 1:2 and added dropwise into chitosan solution by syringe under high-speed stirring by polytron PCU-2-110 high-speed homogenizer (Brinkmann Industries' Westbury, NY, USA). After adjusting pH to 6.1, the mixed solution was maintained under high-speed stirring for another 30 min. To further homogenize and decrease the particle sizes of the insulin particles, they were ultrasonicated for another 30 min using a probe type ultrasonicator (UP 2005T, Hielscher Ultrasonics™, Teltow, Germany). Insulin particles coated with TG-chitosan and MNA-TG-chitosan were prepared as the same method. As this insulin particles would be further processed into buccal tablets, dry insulin particles powder was prepared with a Buchi mini spray dryer B-290™ (BÜCHI™, Flawil, Switzerland) with feeding flow of 3 L/min, and airflow of 4 L/min at 90° C.

### Insulin Particle Characterization

To evaluate the impact of the TG-chitosan and MNA-TG-chitosan on insulin particles. The Z-average diameter, polydispersity index (PDI) and zeta potential of all insulin particles prepared were tested using dynamic light scattering (DLS) measurements using Litesizer 500 (Anton Paar™, Graz, Austria). Morphology and size distribution was characterized by Hitachi H7600™ transmission electron microscopy (TEM) (Hitachi™, Tokyo, Japan), and dry insulin particles were evaluated by Helios NanoLab 650 Focused Ion Beam-Scanning Electron Microscope™ (FIB-SEM) (FEI™, Hillsboro, Oregon, USA)^{37,38}. To evaluate the encapsulation efficiency (EE) and loading content (LC) of insulin particles, the unencapsulated insulin was purified from an ultrafiltration tube and quantified using the Agilent 1100 series™ HPLC system (Agilent™, Santa Clara, California, USA)³⁹. EE and LC in percentages were calculated using Eq. (1) and Eq. (2).

Entrapment efficiency(%)=(^{1-Unencapsulated insulin}/_{Total insulin})×100% (1)

loading content(%(=(^{Weight of encapsulated insulin}/_{Weight of NPs})×100% (2)

All dehydrated insulin particles were re-dissolved in dd water to evaluate their reconstitution abilities. The particle sizes, PDI, EE and LC were tested again using the same methods mentioned before.

### Triple Layer Buccal Tablet Containing Insulin Particles

### Preparation of Triple Layer Buccal Tablets Containing Insulin Particles

To optimize the release profile of insulin along with the mucoadhesion of the tablets while at the same time to achieve uni-directional drug release toward the buccal mucosa, triple layer buccal tablets containing insulin particles were prepared. As shown in TABLE 1, the bioadhesive layer was prepared using 25 mg of the mixture with carbopol, hydroxypropyl methylcellulose, and ethylcellulose. For the purpose of optimizing the mucoadhesion ability, they were prepared in the different ratio (TABLE 1). The insulin loading layer was made of dry insulin particles, mannitol, and sodium alginate (TABLE 1). The resultant powder mixtures were directly compressed into tablets layer by layer using a hand punch tablet press machine (ZONESUN™, Nanhai, China) equipped with 6 mm round and flat punches. All tablets contained an additional water impermeable layer containing 25 mg of ethylcellulose, which was added during the compression stage.

Physical characterization of insulin buccal tablet

The prepared tablets were tested for weight variation, thickness, diameter, insulin content uniformity, Brinell hardness and friability. A weight variation test was performed in compliance with the British Pharmacopoeia™ (Commission, 2012), in which twenty tablets were weighed with a Sartorius CP224 S™ Analytical Balance (Sartorius AG™, Gottingen, Germany). The thickness and diameter of ten tablets were determined by measuring them with a micrometer. Friability tests were performed according to British Pharmacopoeia (2012). In this study, ten tablets were accurately weighed and placed in digital tablet friability tester (BEXCO™, Ambala Cantt, Haryana, India), which was rotated at a speed of 25 rpm for four minutes. It was then calculated the percentage loss in weight as the friabilities. The insulin content uniformity was tested by dissolving 10 tablets in 0.1% of acetic acid after crushing. The insulin quantification was conducted by using HPLC mentioned before. The Brinell hardness was performed with the aid of a spherical indentation probe with a diameter of 3 mm attached to TA.XT plus™ texture analyzer (Stable Micro Systems™, Surrey, UK). After the test has begun, the probe was moved into the sample until it reached a force of 50 N, at which time the load was held for a period of time, and then the probe was completely withdrawn.

### Surface pH

Before the surface pH test, bioadhesive layers of all tablets were wetted and kept in contact with 1 ml of artificial saliva at room temperature for two hours. In this test, the pH of the tablet was measured by placing the electrode of the Fisherbrand Accumet AE150™ Benchtop pH Meter (Waltham, Massachusetts, USA) in contact with the surface of the tablet. The electrode was then allowed to equilibrate on the tablet for one minute. The surface pH of each tablet was determined in triplicate, and the mean and standard deviation for each were calculated.

TABLE 1
The composition of the insulin-loaded triple-layer buccal tablet

| | | | | | | | | | |
|---|---|---|---|---|---|---|---|---|---|
| Excipients (mg) | F1 | F2 | F3 | F4 | F5 | F6 | F7 | F8 | F9 |

Mucoadhesive Layer

| | | | | | | | | | |
|---|---|---|---|---|---|---|---|---|---|
| Carbopol 934P | | 3.125 | 6.25 | | 4.165 | 8.33 | | 6.25 | 12.5 |
| HPMC | 6.25 | 3.125 | | 8.33 | 4.165 | | 12.5 | 6.25 | |
| Ethylcellulose | 18.75 | 18.75 | 18.75 | 16.67 | 16.67 | 16.67 | 12.5 | 12.5 | 12.5 |

Peptide Loading Layer

| | | | | | | | | | |
|---|---|---|---|---|---|---|---|---|---|
| Insulin particle | 5 | 5 | 5 | 5 | 5 | 5 | 5 | 5 | 5 |
| Mannitol | 40 | 40 | 40 | 40 | 40 | 40 | 40 | 40 | 40 |
| Sodium alginate | 5 | 5 | 5 | 5 | 5 | 5 | 5 | 5 | 5 |

Water Impermeable Layer

| | | | | | | | | | |
|---|---|---|---|---|---|---|---|---|---|
| Ethylcellulose | 25 | 25 | 25 | 25 | 25 | 25 | 25 | 25 | 25 |

### Swelling Index Study

This study was carried out to determine the swelling index for each tablet, and then the mean ±SD was calculated. Each insulin buccal tablet was weighted (W₀), placed separately on the 2% agar gel plates, and incubated at 37±1° C. Throughout the experiment, the tablet was removed from the petri dish at regular intervals until eight hours, and the surface water was carefully removed with filter paper. After weighing the swollen tablet (W₁) a second time, the swelling index was calculated using the following equation (Eq. 3):

Swelling index(%)=(^{W1-W0}/_{W0})×100%

### Ex-vivo Mucoadhesion Strength Measurement

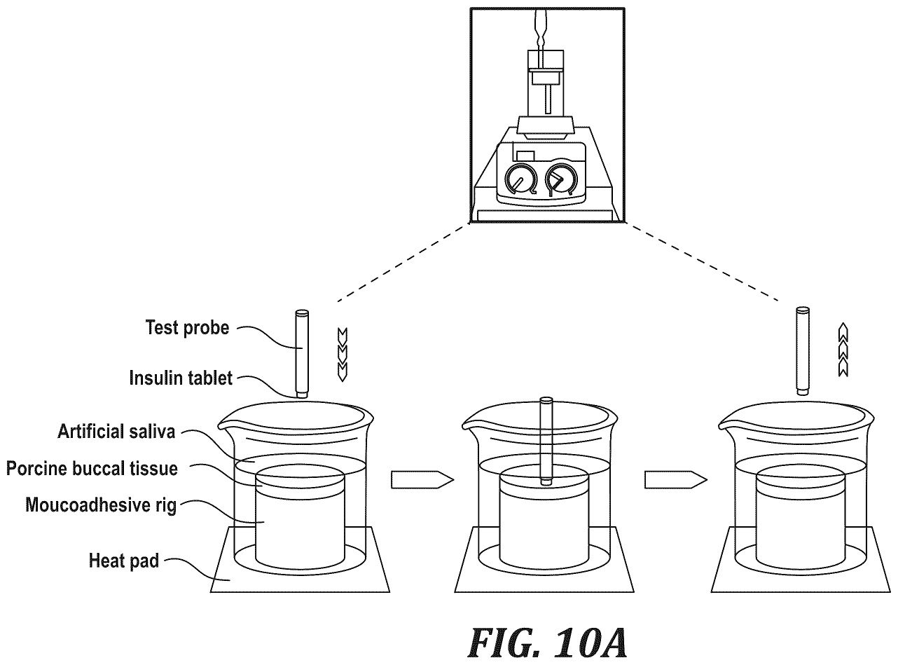

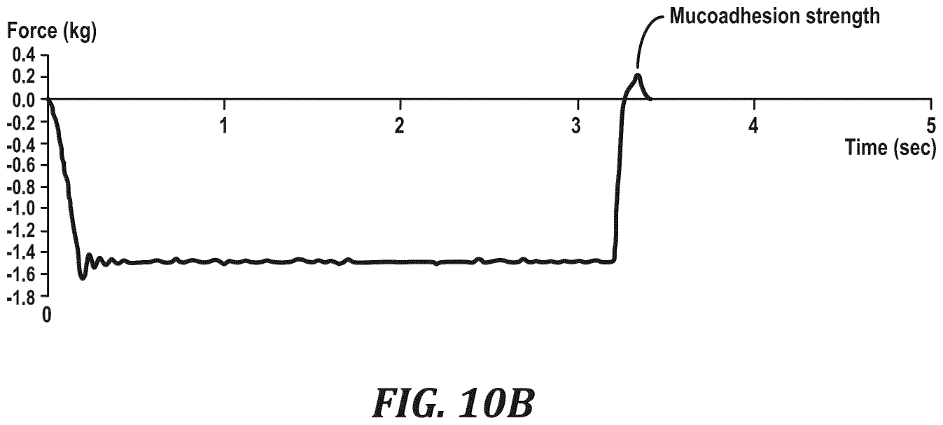

The mucoadhesive forces between the buccal mucosa and insulin buccal tablets were assessed in a detachment test using a TA-XT plus texture analyzer (Stable Micro Systems™, Surrey, UK) with a mucoadhesion rig (A/MUC). Freshly excised porcine buccal tissue was obtained from UBC animal care center. The buccal tissue was attached to the mucoadhesion test rig (FIG. 10). To mimic the condition in mouth, mucoadhesion test rig was put in a beaker filled with artificial saliva (FIG. 10). The water level of the artificial saliva was just in contact with the porcine buccal tissue (FIG. 10). During the test, a stirring rate of 150 rpm was applied and the temperature was set up at 37° C. The probe, which was fixed with insulin tablets, moved at a constant speed of 1 mms·s⁻¹ toward the surface of the porcine buccal tissue and was kept in contact for 30 seconds with no force applied during this interval. After 30 seconds, the probe with the tablets was drawn upward (0.5 mms·s⁻¹) until the contact between the surfaces was broken. The mucoadhesive forces of the tablets were evaluated by measuring the maximum force required to detach the probe, which directly indicated the mucoadhesion strength.

### Ex-vivo Penetration Study

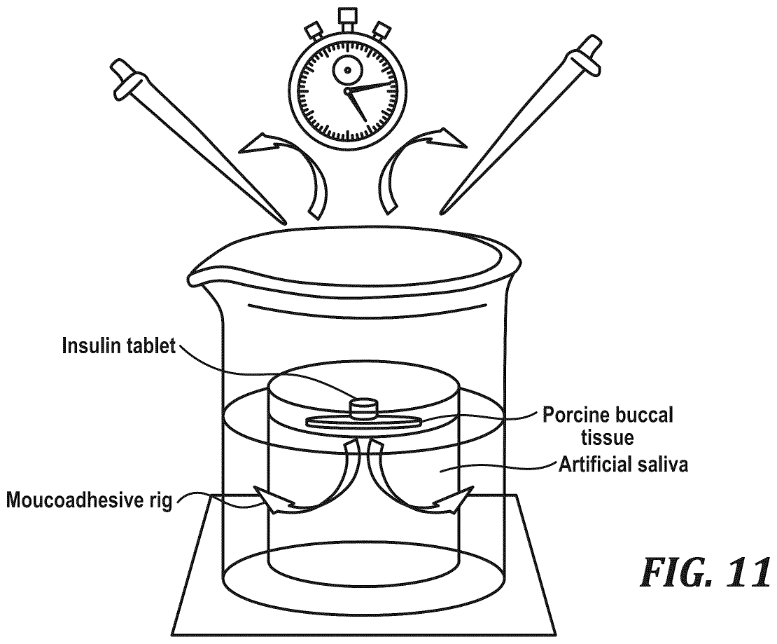

The penetration study of insulin buccal tablets was carried out with the assistance of the mucoadhesion rig. The buccal tissue was cut into a section measuring 2 mm thick and attached to the mucoadhesion test rig (FIG. 11). The setup was similar to the mucoadhesion strength measurement (FIG. 11). To test the uni-directional release profile of the tablet, both sides of the tablets were attached to porcine buccal tissue and tested for their penetration behaviors. At the time point of 0.5, 1, 2, 3 and 4 h, 0.5 mL of the fluid in the beaker were withdrawn and the volume immediately replenished with fresh artificial saliva. The fluid was analyzed for insulin contamination by the human insulin ELISA Kit (Abcam™, Toronto, Ontario, Canada) according to the manufacturer's instructions. The penetrated insulin through buccal tissues was calculated from the ratio of penetrated insulin to the total insulin in the tablets (Eq. 4).

Insulin penetration rate(%)=(^{Weight of release insulin}/_{Weight of insulin in tablets})×100% (4)

In vitro Cell Studies—Cell Cultivation

### HepG2 Cells

HepG2 cells, human hepatocellular carcinoma cell line, were cultivated in Nunc™ cell culture petri dishes (Thermo Fisher™, NY, USA) with 60 mm diameter using Dulbecco's Modified Eagle Medium (DMEM) containing 10% of fetal calf serum, 100 IU/mL of penicillin and 100 μg/mL of streptomycin. The culture was kept at the environment of 37° C., 95% relative humidity with 5% CO₂. Media were changed every 2-3 days depending on the growth rate. Cells were seeded at 2×10⁴ cells/cm² for sub-cultivation twice a week using 0.25% trypsin-EDTA.

### Caco 2 Cells

Caco 2 cells, widely used as a model of the intestinal epithelial barrier, were cultivated in Nunc™ cell culture petri dishes (Thermo Fisher™, NY, USA) with 60 mm diameter using

Dulbecco's Modified Eagle Medium (DMEM) containing 10% of fetal calf serum, 100 IU/mL of penicillin and 100 μg/mL of streptomycin. The culture was kept at the environment of 37° C., 95% relative humidity with 5% CO₂. Media were changed every 2-3 days depending on the growth rate. Cells were seeded at a concentration of 2×10⁴ cells/cm² for sub-cultivation once a week using 0.25% trypsin-EDTA.

### TR 146 Cells

TR146 cells, appropriate for drug transport studies and mimic normal human buccal epithelium, were cultivated in Nunc™ cell culture petri dishes (Thermo Fisher™, NY, USA) with 60 mm diameter using Nutrient Mixture F-12 Ham containing 10% of fetal calf serum, 100 IU/mL of penicillin and 100 μg/mL of streptomycin. The culture was kept at the environment of 37° C., 95% relative humidity with 5% CO₂. Media were changed every 2-3 days depending on the growth rate. Cells were seeded at 2×10⁴ cells/cm² for sub-cultivation once a week using 0.25% trypsin-EDTA.

In vitro Cytotoxicity Assay

The MTT test was used to assess the cytotoxicity of insulin particles coated with chitosan, TG-chitosan and MNA-TG-chitosan after reconstitution. The HepG2 cells, Caco 2 cells and TR-146 cells were seeded at a density of 5×10⁴, 5×10⁴ and 1×10⁴ cells/cm 2 in 96 well plates. Insulin particles were diluted to various concentrations (50 to 1000 μg/mL) in their respective culture medium and then were given to the cells. After 8 h incubation, the cells were washed with PBS three times and refreshed with a medium containing 0.5 mg/ml of MTT for another 4 h incubation. The cytotoxicity was evaluated by measuring the enzymatic reduction of yellow tetrazolium MTT to purple formazan at 570 nm using Tecan Infinite M200™ pro spectrophotometer plate reader (Tecan™, Mannedorf, Switzerland).

In vitro Cellular Uptake Assay

The insulin particles cellular uptake efficacy was tested by confocal laser scanning microscope and flow cytometry analysis. The HepG2 cells were seeded at a density of 5×10⁴ cells/cm² in Nunc Lab-Tele™ chamber slide system. Each well of Nunc Lab-Tek chamber slide system was treated with free FITC-insulin, FITC-insulin particles coated with chitosan, TG-chitosan and MNA-TG-chitosan at the same concentration of 25 μg/mL and incubated for four h. Cells were then fixed with 4% paraformaldehyde and the nuclei of the cells were stained with DAPI. The localization of insulin was observed using an Olympus FV1000™ laser scanning/two-photon Confocal Microscope (Olympus™, Shinjuku City, Tokyo, Japan).

For flow cytometry analysis, HepG2 cells were seeded at a density of 5×10⁴ cells/cm² in 96 well plates. Free FITC-insulin, FITC-insulin particles coated with chitosan, TG-chitosan and MNA-TG-chitosan with a concentration of 10 μg/mL were added into the 96-well plate and incubated for 4 h. After 4 h incubation, cells were lifted and washed three times with FBS. 5×10⁴ cells per sample were analyzed by BD LSR II™ flow cytometer (BD™, Franklin Lakes, New Jersey, USA).

In vitro Penetration Test

To evaluate permeability, insulin particles were tested using Corning™ (Transwell pore diameter 0.4 μm, growth area 0.33 cm²) inserts with a 24-well plate. In brief, TR146 cells were cultured on the insert for 30 days at a density of 5×10⁴ cells/cm², with the medium being changed every two days. Free insulin, and insulin particles coated with chitosan, TG-chitosan and MNA-TG-chitosan were tested in 24-well plate, respectively. From the receptor portion, 0.1 mL of the sample was taken at 0.5, 1, 2, 3, 4, 6 h. The receptor portion was immediately refilled with fresh PBS in order to maintain the sinking conditions.

### Cell Tight Junction Test—-TEER Value Test

By studying the changes in transepithelial electrical resistance (TEER) values of a monolayer of epithelial cells after the insulin particles treatment, the opening of tight junctions has been studied. This test was carried out using Corning™ (Transwell pore diameter 0.4 μm, growth area 0.33 cm²) inserts with a 24-well plate. TR146 cells were cultured on the insert for 30 days at a density of 5×10⁴ cells/cm². The cells were incubated with free insulin, insulin particles coated with chitosan, TG-chitosan and MNA-TG-chitosan, respectively and the changes in TEER values were measured with a Millicell™-Electrical Resistance System™ at different time intervals within 4 hours (Millipore™, MA, USA). After incubation, the test sample was removed and TEER values were monitored for another 20 h.

### Visualization of Tight Junctions

The status of tight junction between the TR146 cells after the incubation with NPs was visualized by the immunofluorescent staining of ZO-1 protein. The TR146 cells were seeded at a density of 5×10⁴ cells/mL in Nunc Lab-Tele™ chamber slide system. The TR146 cell monolayer was incubated with free insulin, insulin particles coated with chitosan, TG-chitosan and MNA-TG-chitosan for 4 h, and then the cells were fixed with 4% paraformaldehyde. The cells were then permeabilized with 0.1% Triton X-100, and blocked with 5% goat serum. Subsequently, cells were treated with ZO-1 monoclonal antibody (Thermo Fisher™, NY, USA) followed by Goat anti-Mouse IgG (H+L) Cross-Adsorbed Secondary Antibody, FITC (Thermo Fisher™, NY, USA). The stained cells were washed three times with PBS, mounted on slides, and visualized using Olympus FV1000™ laser scanning/two-photon Confocal Microscope (Olympus™, Shinjuku City, Tokyo, Japan).

### In vivo Studies

### Diabetic Rat Model

Female Wistar rats weighing 600±30 g were purchased From The University of British Columbia Animal Care Service. Type I diabetes was induced by a single intravenous (i.v.) injection of streptozotocin (STZ) at 55 mg/kg 4 days before the pharmacodynamic tests. The body weight and blood glucose were checked at least every 24 hours after the injection via tail poke. The bold glucose was evaluated using a OneTouch Verio Reflect™ meter (OneTouchT™, Surry, British Columbia, Canada) and strips purchased from the pharmacy. The rats were considered to have diabetes once their glycemia had reached a level higher than 16.0 mM. Before performing the experiment, the insulin resistance of the rats was determined to avoid giving the wrong dose of insulin. About 2 IU/kg of insulin was used to test the insulin resistance of the rats.

Hypoglycemic Effect In vivo

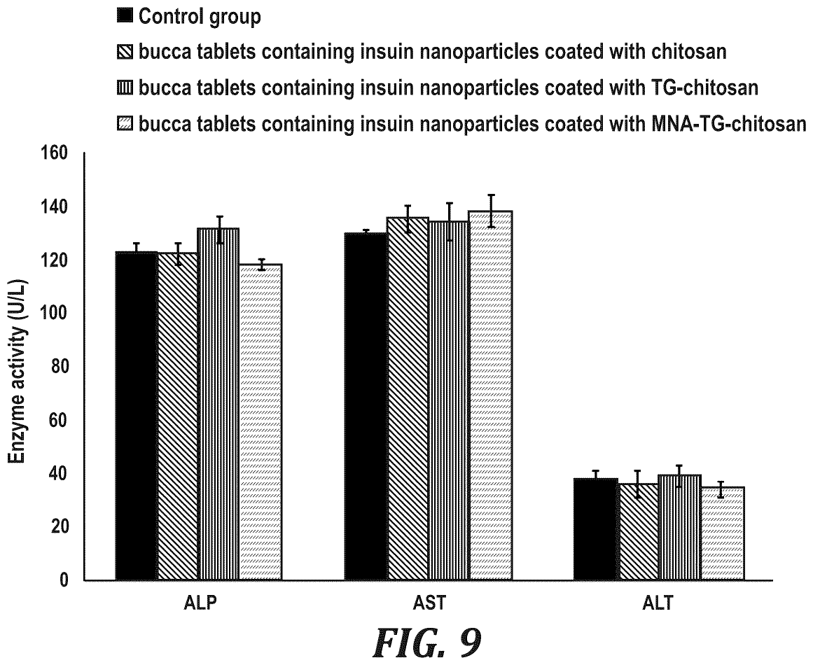

Free insulin solution and insulin particles coated with chitosan, TG-chitosan and MNA-TG-chitosan were administrated at a dose of 50 IU/kg via oral gavage. I.p. injection of the dose of 10 IU/kg insulin was used in this study as a positive control. Other groups of diabetic rats were administrated buccal tablets with insulin and insulin particles coated with chitosan, TG-chitosan and MNA-TG-chitosan respectively under anesthesia (FIG. 9) with an insulin concentration of 50 IU/kg. During the experiments, rats fasted with free access to water and blood glucose level was determined by sampling the blood from the tail vein with a glucometer at predetermined time points.

### Biodistribution of Orally Delivered Insulin Particles and Insulin Buccal Tablets

This study was carried out using female Wistar rats weighing 600±30 grams. The particle biodistribution was investigated using Cy-5 labeled insulin prepared according to previous research⁴⁰. The Cy-5 labeled insulin was prepared into MNA-TG-chitosan coated particles and further made into buccal tablets containing it. The Cy-5 labeled insulin solution was given through i.p. injection and the Cy-5 labeled insulin particles solution was administrated via oral gavage. The buccal tablets containing Cy-5 labeled insulin particles was given under anesthesia using the same method mentioned before. The global distribution of Cy5-labeled insulin was revealed by near infrared imaging using an IVIS Lumina II in vivo optical imaging system (PerkinElmer™, Waltham, Massachusetts, USA). The images were taken after two hours of insulin administration of all rats. All rats were euthanized and shaved before the imaging.

### Pharmacokinetics Assessment

The pharmacokinetics of insulin have been evaluated in rats that have been induced to become diabetic by STZ as described previously. Free insulin, insulin particles and insulin buccal tablets were given using the same method mentioned in the “In vitro cytotoxicity assay” above. Serum insulin level was quantified using human insulin ELISA Kit (Abcam™, Toronto, Ontario, Canada) according to the manufacturer's instructions. By comparing the area under the curve for the insulin level profile of those taking oral gavage or tablets with those taking direct i.p. injection, the relative bioavailability of insulin was calculated. The calculation is shown in Equation 5.

Relative bioavailability(=(^{AUCoral×Doseip}/_{AUCip×Doseoral})×100% (5)

While AUCₒᵣₐₗ and AUCᵢₚ referred to the total area under the curve of the serum insulin concentration in either the oral gavage or tablets and by i.p. injection, respectively. Doseₒᵣₐₗ and Doseᵢₚ represented the dose of insulin used of either the oral gavage or tablets, and by i.p. injection, respectively. Comparative analysis among all groups was performed in Graph-Pad Prism 9.4.0™ for Mac OS (GraphPad Software™, San Diego, California, USA).

Toxicity In vivo

Biochemical indicators were tested using alkaline phosphatase (ALP) assay kit (ab83369 or ab83371), aspartate aminotransferase (AST) assay kit (ab105135), and alanine transaminase (ALT) assay kit (ab105134) in serum were obtained from Abcam™ (Toronto, Ontario, Canada) according to the manufacturer's instructions. These assays were used to determine whether buccal tablets containing insulin particles coated with chitosan, TG-chitosan and MNA-TG-chitosan were safe to use. Rats were treated with a daily dose of 50 IU/kg insulin buccal tablets and after one day, they were euthanized, and the serum was used for toxicity analysis.

### Statistical Analysis

All experiments were performed in triplicates and values are expressed as Mean±SD. Comparisons among all groups were evaluated using One-way ANOVA or t-test by IBM SPSS Statistics 26 for Mac (IBM™, Endicott, New York, USA), and p<0.05 was considered to be statistically significant.

### EXAMPLES

### Example 1

### Characterization of TG-chitosan and MNA-TG-chitosan

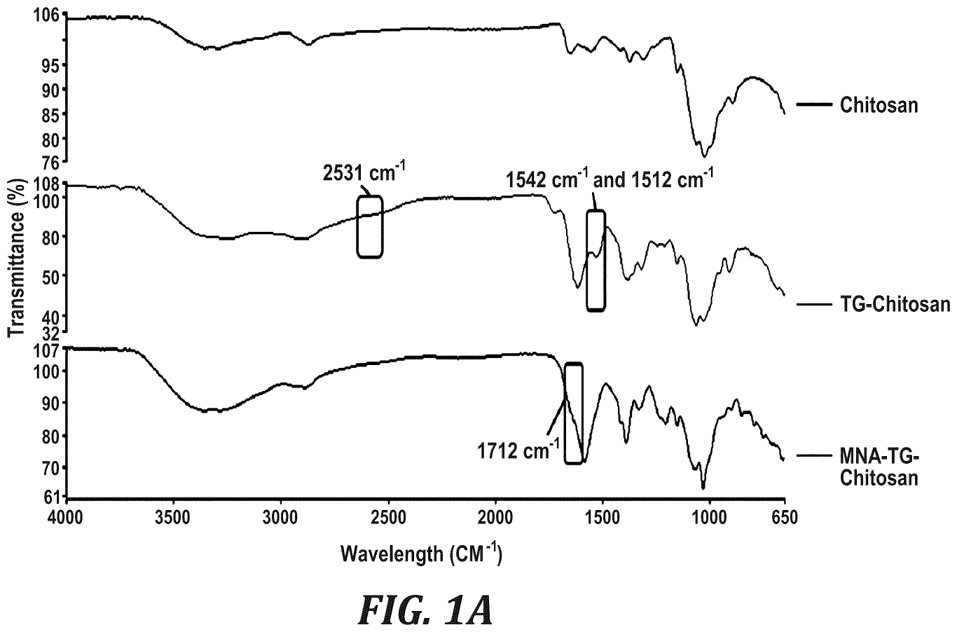

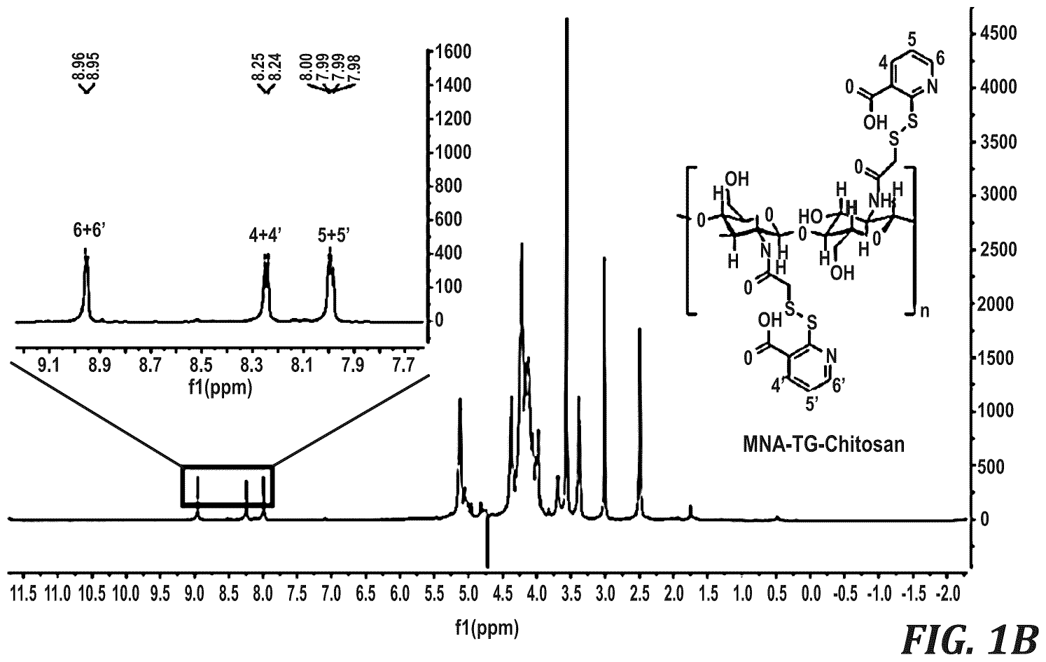

TG-chitosan conjugate had a thiol group content of 218±33 μmol per gram of the polymer. ATR-FTIR spectra of chitosan (FIG. 1A) showed the absorption peaks at 1665 and 1622 cm⁻¹, respectively, which corresponds to the characteristic amide I band observed in both chitosan and TG-chitosan¹⁴. The peaks at 1542 cm⁻¹ and 1512 cm⁻¹ identified in both chitosan and TG-chitosan are due to amide II bands (CN stretching and NH bending)¹⁴. An absorption band at 3303 cm⁻¹ and 3262 cm⁻¹ is attributed to NH stretch, and a weak peak at 2531 cm⁻¹ indicates the conjugation of chitosan and TGA¹⁴.

Moreover, by calculating the content of total and free thiol groups through Ellman's assay, the preactivated MNA-TG-chitosan exhibited 138±21 μmol of disulfide bond per gram of polymer. This indicates more than 50% of the thiol groups have been preactivated, consistent with the previous result based on preactivated MNA-TG-cellulose¹⁵. In addition, preactivated MNA-TG-chitosan was also evaluated using FT-IR spectra. FIG. 1A showed the preactivated MNA-TG-chitosan structure, which can be recognized by the NH bend detected at 1653-1619 cm⁻¹. The presence of amide bonds in the MNA-TG-chitosan was found in contrast to the unmodified chitosan in the 1712 cm⁻¹¹⁵. The NMR data can be seen in FIG. 1B, while the coupling reagent disulfandiyldinicotinic acid was detected there. Given the pure spectra and the presence of disulfide bonds in the MNA-TG-chitosan, it could be proved that the synthesis of preactivated MNA-TG-chitosan has been conducted in this study.

### Example 2

### Characterization of Insulin Particle and Impact of TG-chitosan and MNA-TG-chitosan

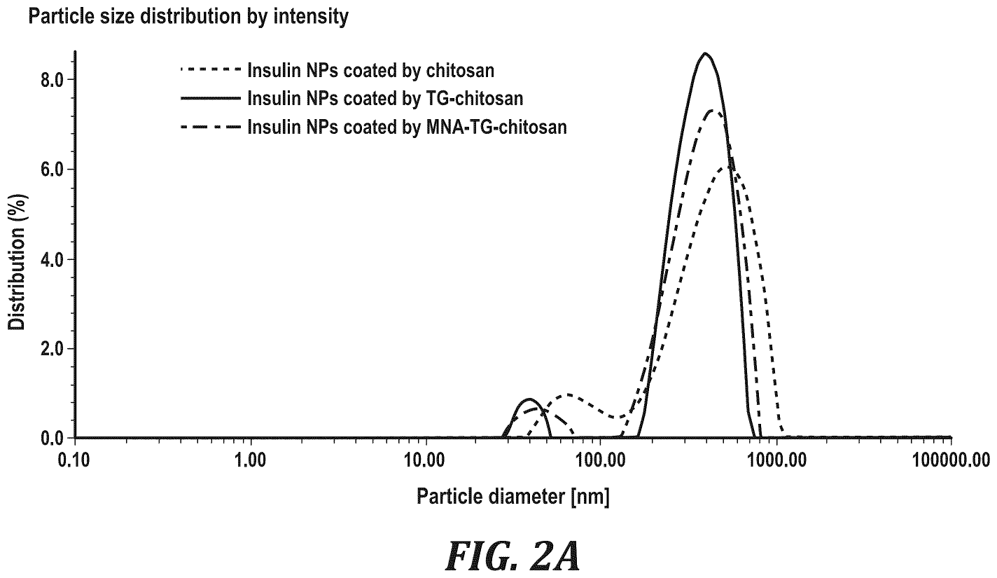

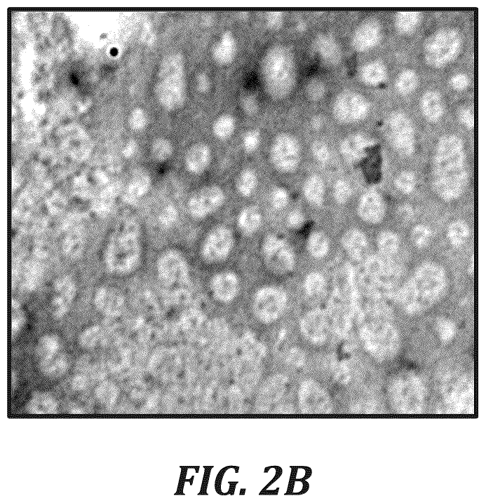

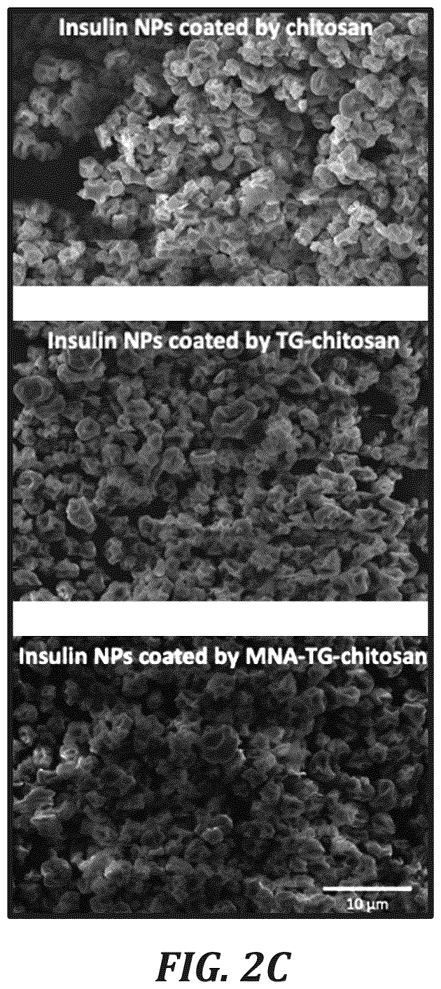

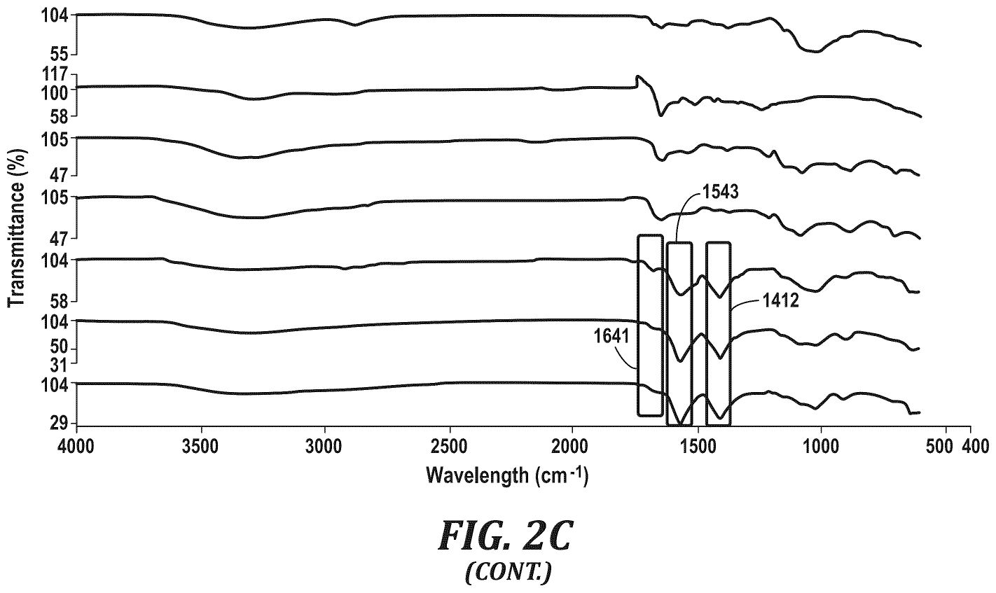

The insulin particles coated with chitosan were prepared using the optimized condition from our previous study¹⁰. The optimized insulin particles resulted in 318 nm of mean particle size, 0.18 of PDI, 99.03% of entrapment efficiency, 9.8 my of zeta potential, and 25.19% (m/m) of insulin loading content (TABLE 2A). Based on the transmission electron microscope (TEM) results, the optimized particles were spherical and discrete with relatively uniform size (FIG. 2B). SEM results suggested the spherical structure with a wrinkled surface after spray drying without any bulking agents (FIG. 2C). After reconstitution, the mean particle size of the insulin particles coated with chitosan was increased to 457 nm, while the PDI, EE, and loading content did not significantly change (p<0.05). Given that this particular MNA-TG-chitosan has been synthesized, its impact as a coating material on insulin particles has also been investigated. Using the same optimized preparing condition, the mean particle size of insulin particles coated with TG-chitosan decreased more than insulin particles coated with MNA-TG-chitosan with no change in PDI, EE, and loading content (TABLE 2A). The morphology of the dehydrated insulin particles coated with TG-chitosan and MNA-TG-chitosan had no visible changes as the insulin particles coated with chitosan (FIG. 2C). The reconstitution ability of the dehydrated insulin particles coated with TG-chitosan and MNA-TG-chitosan also showed smaller particle sizes than the insulin particles coated with non-thiolated chitosan (FIG. 2A). All these results indicated that the MNA-TG-chitosan had no negative effects on insulin NPs characterization. Moreover, a previous study proved that the TG-chitosan could significantly decrease the mean particle sizes of particles because of the formation of —S—S- bonds in or outside the particles^{16,17}, which was consistent with our results. However, in our case, most of the —SH groups of MNA-TG-chitosan have been connected to the MNA group (Synthesis 1). Therefore, only part of them can form —S—S- bonds outside the particles so that it can slightly decrease mean particle size and have more —SH groups to increase its bioadhesive ability. Free insulin, chitosan, physical mixture of chitosan, TPP and insulin and all dehydrated NPs were characterized by using ATR-FTIR spectroscopy. Noticeably, increases in the band intensities at 1641, 1543 and 1412 cm⁻¹ were observed in encapsulated particles freeze-dried with mannitol and particles spray-dried both with and without mannitol (FIG. 2D). These increases in intensities are associated with the cross link among chitosan, TPP and insulin. After reconstitution in water, the encapsulation efficiency (EE) of all the particles slightly decreased, and around a small amount (˜5%) of insulin was released during the three-month storage (TABLE 2B). However, the mean particle size of all the particles increased. The particle size of the particles that were spray dried without mannitol was increased to 525 nm while the particles size of the spray-dried and freeze-dried particles with mannitol increased to 872 and 921 nm, respectively (TABLE 2B).

TABLE 2A
Physicochemical properties of freshly
prepared and reconstituted particles

| | | | Insulin coated |
|---|---|---|---|
| | Insulin coated | Insulin coated | with MNA-TG- |
| | with chitosan | with TG-chitosan | chitosan |
| Z-average | 318 ± 18 | 235 ± 16 | 277 ± 14 |
| diameter (nm) | | | |
| Polydispersity index | 0.18 ± 0.01 | 0.13 ± 0.03 | 0.21 ± 0.02 |
| Encapsulation | 99.03 ± 0.33^{a} | 98.43 ± 0.64^{a} | 98.79 ± 0.43^{a} |
| efficiency (%) | | | |
| Loading content (%) | 25.19 ± 0.37^{a} | 25.35 ± 0.53^{b} | 25.03 ± 0.43^{a} |

After reconstitution

| | | | |
|---|---|---|---|
| Z-average | 457 ± 34 | 323 ± 29 | 389 ± 33 |
| diameter (nm) | | | |
| Polydispersity index | 0.21 ± 0.04 | 0.19 ± 0.05 | 0.21 ± 0.03 |
| Encapsulation | 98.09 ± 0.21 | 98.31 ± 0.39 | 98.43 ± 0.53 |
| efficiency (%) | | | |
| Loading content (%) | 24.87 ± 0.43 | 25.02 ± 0.26 | 24.92 ± 0.52 |

TABLE 2B
Physicochemical properties of particles after three-month storage

| | particles | | |
|---|---|---|---|
| | freeze | NPs spray | NPs spray |
| | dried with | dried without | dried with |
| | mannitol | mannitol | mannitol |
| Z-average | 921 ± 102^{a} | 525 ± 77^{b} | 872 ± 117^{c} |
| diameter (nm) | | | |
| Polydispersity | 0.25 ± 0.07^{a} | 0.23 ± 0.02^{a} | 0.26 ± 0.07^{a} |
| index (PDI) | | | |
| Encapsulation | 91.36 ± 0.73^{a} | 92.00 ± 0.88^{a} | 92.29 ± 0.67^{a} |
| efficiency (%) | | | |
| Loading content (%) | 5.06 ± 0.32^{a} | 23.02 ± 0.41^{b} | 5.08 0.22^{a} |

### Example 3

### Physical Characterization of Triple Layer Buccal Tablets

All tablets containing insulin particles were tested for content uniformity, weight variation, friability, and Brinell hardness. The average insulin content in different formulations ranged from 49.37±0.82 to 50.83±0.49 IU. The average weight of all prepared tablets ranged from 98.37±1.32 to 101.21±1.41 mg. In the friability test, there were no physical signs of cracking, cleaved or broken tablets after the test, and the average weight loss was from 0.21±0.12 to 0.29±0.09%. The average thickness of all tablets ranged from 3.91±0.12 to 4.10±0.14 mm, with 6.00±0.02 mm diameters. The Brinell hardness is a guide to the material's resistance to wear and deformation and its ability to indent or abrade another material¹⁸. In this study, all tablets exhibited the Brinell hardness of around 1.8 kg/mm² (data not shown). Based on the results, insulin buccal tablets with all formulations had acceptable content uniformity, weight variation, friability, hardness, and size distribution. Moreover, the involvement of the insulin particles coated with chitosan, TG-chitosan, and MNA-TG-chitosan did not change the physical characterization of the prepared buccal tablets. Thus, other studies can use all formulations to get the optimized insulin buccal tablets.

### Example 4

### Surface pH Study

As acidic or alkaline pH is correlated to irritate the buccal mucous membrane in the oral cavity, the surface pH of tablets was determined. The surface pH of the adhesive layers of all tablets was from 5.44±0.20 to 6.12±0.28. Based on these results (data not shown), it is concluded that all formulations provided an acceptable pH level within the salivary pH range (5.5-7.0) and would not produce any local irritation on the mucosal surface when applied¹⁹. Moreover, the involvement of the insulin particles coated with chitosan, TG-chitosan, and MNA-TG-chitosan did not change the surface pH of the prepared buccal tablets as they were all located in the middle layer of the tablets.

### Example 5

### Swelling Study

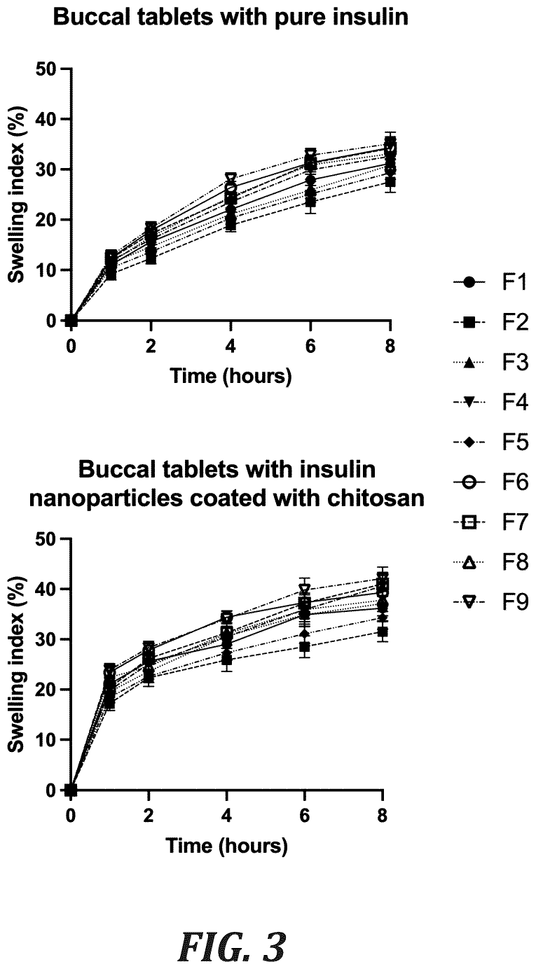

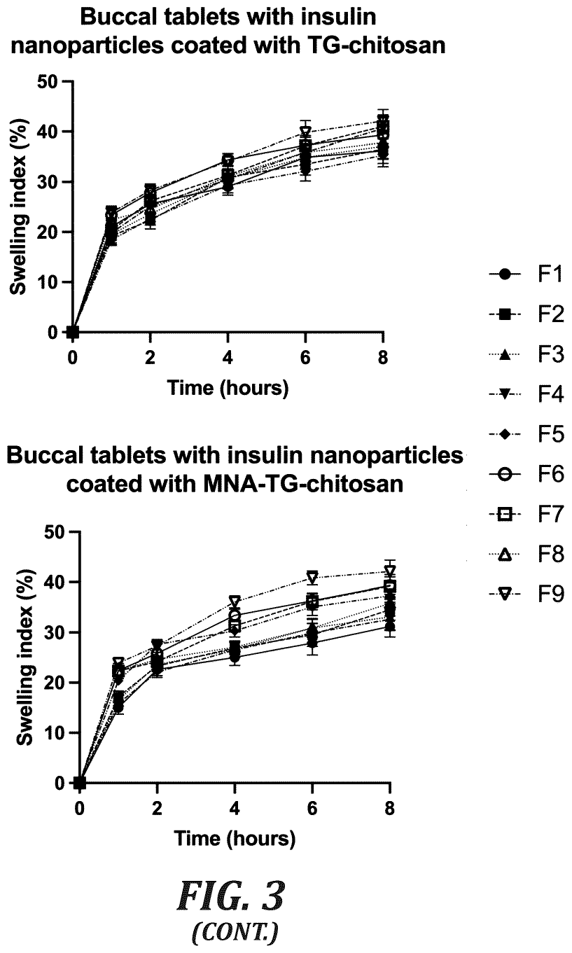

The buccal adhesive tablet should have an appropriate swelling behavior to facilitate a prolonged release of the drug and effective mucoadhesion to the mucosa²⁰. As shown in FIG. 3, tablets containing insulin chitosan encapsulated particles showed slightly higher swelling indices than those with pure insulin. However, tablets containing insulin-loaded

TG-chitosan, and MNA-TG-chitosan particles did not show higher swelling indices than those containing insulin-loaded chitosan particles (FIG. 3C and D). The high rate and extent of swelling of formulations with three kinds of chitosan particles could be attributed to the high amount of water uptake by these three polymers along with their fast swelling properties. Moreover, as TG-chitosan and MNA-TG-chitosan did not change the water-uptake nature of chitosan, the tablets with those two materials showed similar swelling properties. Compared with all the formulations, F9 exhibited the highest swelling index by the end of the 8 hour test (FIG. 3). Compared with hydroxypropyl methylcellulose, carbopol 934P™ has higher water solubility²¹, whereby the water penetrates more quickly than the tablets containing hydroxypropyl methylcellulose. As for F3 and F6, although only carbopol 934P™ is found in the adhesive layer, the decrease in the swelling compared to F9 might be attributed to the presence of high content of low swelling and water-insoluble polymer polymers of ethylcellulose.

### Example 6

### Ex-vivo Evaluation of Triple Layer Buccal Tablets Containing Insulin Particles—Mucoadhesion Strength

According to TABLE 3, different insulin buccal tablets with different adhesive layers had different mucoadhesion forces. The tablets with F9 showed the strongest mucoadhesion force (1.12±0.16 N), whereas the tablets with F1 showed the weakest mucoadhesion force (0.09±0.01 N). Using chitosan, TG-chitosan, and MNA-TG-chitosan-coated insulin particles did not increase the mucoadhesion strength of the tablets. This is probably because the insulin particles were located in the middle layer of the buccal tablets and thus did not contribute to mucoadhesion, as much as the mucoadhesive layer.

Carbopol 934P™ had the highest contribution on the mucoadhesion forces since F9, F6 and F3 showed the highest mucoadhesion forces compared with other formulations with the same content of ethylcellulose (TABLE 3). The analysis of the ratio between ethylcellulose and other materials shows that ethylcellulose contributed least to mucoadhesion, since greater amounts of ethylcellulose showed low mucoadhesion forces. The mucoadhesion force of different polymers in this study could be as follows: carbopol 934P™ >hydroxypropyl methylcellulose>ethylcellulose. This ranking was comparable to previous studies²². Due to the higher number of carboxylic groups present in carbopol 934P™, it has a higher mucoadhesion strength due to the formation of secondary mucoadhesion bonds with mucin through hydrogen bonds. There was also evidence that when carbopol 934P™ chains were exposed to mucus with higher concentration, the interpenetration of carbopol 934P™ into mucus is increased, while the other polymers are able to provide superficial mucoadhesion²³.

The reason hydroxypropyl methylcellulose has a weaker mucoadhesion force compared with carbopol 934P™ could be due to the absence of proton-donating carboxyl groups in its structure, which reduce its ability to form hydrogen bonds. Furthermore, ethylcellulose was found to have the lowest contribution to adhesion. Increasing the ratio of ethylcellulose and other materials from 1:1 to 2:1 to 3:1 significantly decreased the mucoadhesion force (TABLE 3). This result was in agreement with the previous study, as well¹⁵, where ethylcellulose attributed to the lack of the physical integrity of the formed gel layer and the lowest hydration ability compared with other materials used in that study.

TABLE 3
Mucoadhesion forces of insulin buccal tablets

| | | | | Tablets |
|---|---|---|---|---|
| | | Tablets | Tablets | with insulin |
| | | insulin | with insulin | particles |
| Mucoad- | Tablets | particles | particles | coated with |
| hesion | with pure | coated with | coated with | MNA-TG- |
| forces (N) | insulin | chitosan | TG-chitosan | chitosan |
| F1 | 0.10 ± 0.03 | 0.09 ± 0.02 | 0.09 ± 0.01 | 0.10 ± 0.01 |
| F2 | 0.34 ± 0.08 | 0.36 ± 0.05 | 0.29 ± 0.05 | 0.32 ± 0.04 |
| F3 | 0.63 ± 0.04 | 0.62 ± 0.07 | 0.65 ± 0.03 | 0.59 ± 0.06 |
| F4 | 0.13 ± 0.02 | 0.15 ± 0.02 | 0.14 ± 0.04 | 0.16 ± 0.03 |
| F5 | 0.50 ± 0.03 | 0.53 ± 0.03 | 0.52 ± 0.07 | 0.55 ± 0.02 |
| F6 | 0.88 ± 0.08 | 0.91 ± 0.03 | 0.92 ± 0.10 | 0.86 ± 0.08 |
| F7 | 0.19 ± 0.03 | 0.18 ± 0.05 | 0.21 ± 0.05 | 0.17 ± 0.04 |
| F8 | 0.82 ± 0.10 | 0.91 ± 0.04 | 0.94 ± 0.13 | 0.76 ± 0.11 |
| F9 | 1.09 ± 0.11 | 1.12 ± 0.16 | 1.11 ± 0.09 | 1.11 ± 0.19 |

### Example 7

### Ex-vivo Evaluation of Triple Layer Buccal Tablets Containing Insulin Particles—Ex-Vivo Penetration Test

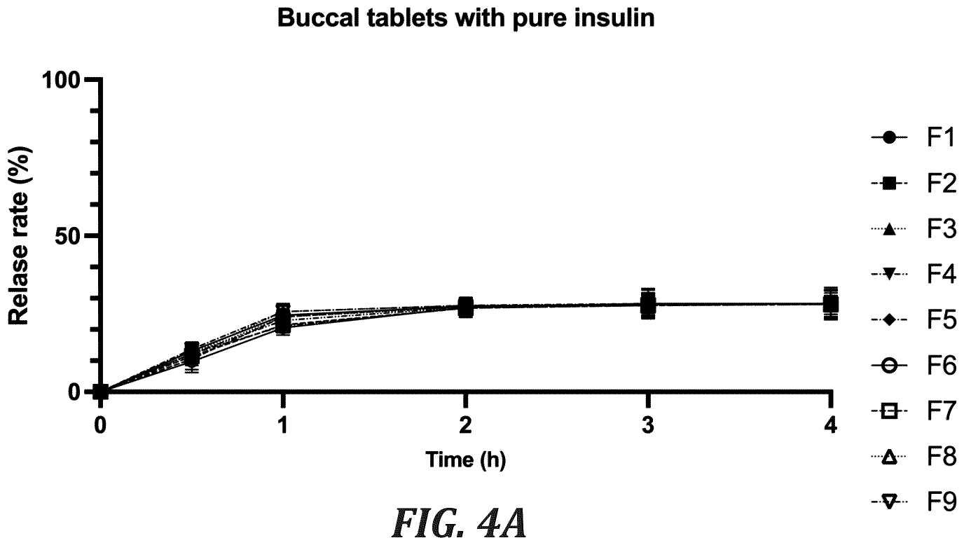

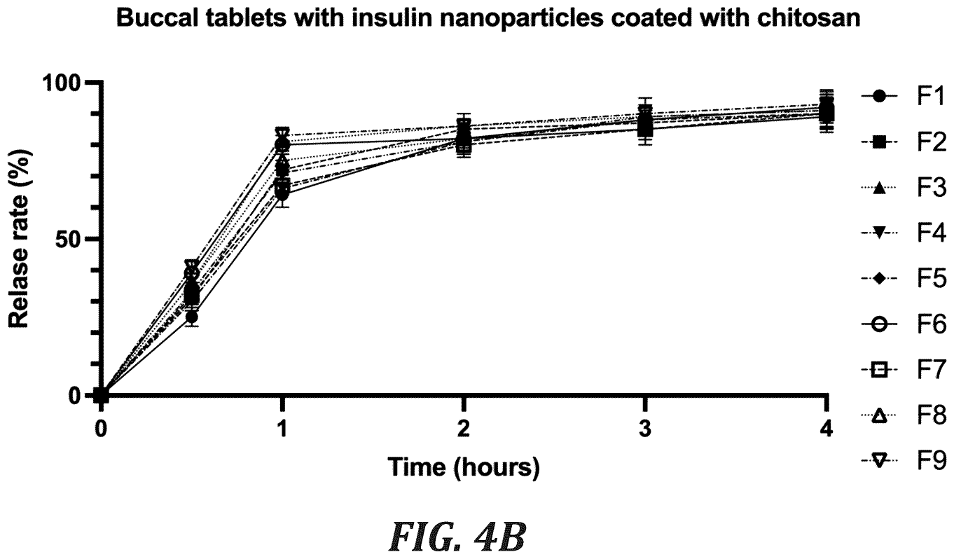

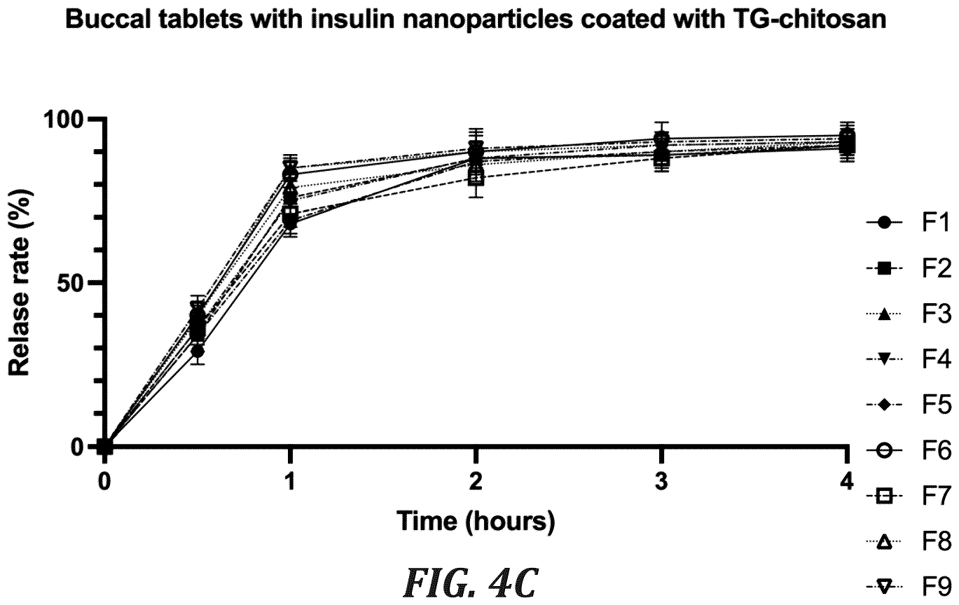

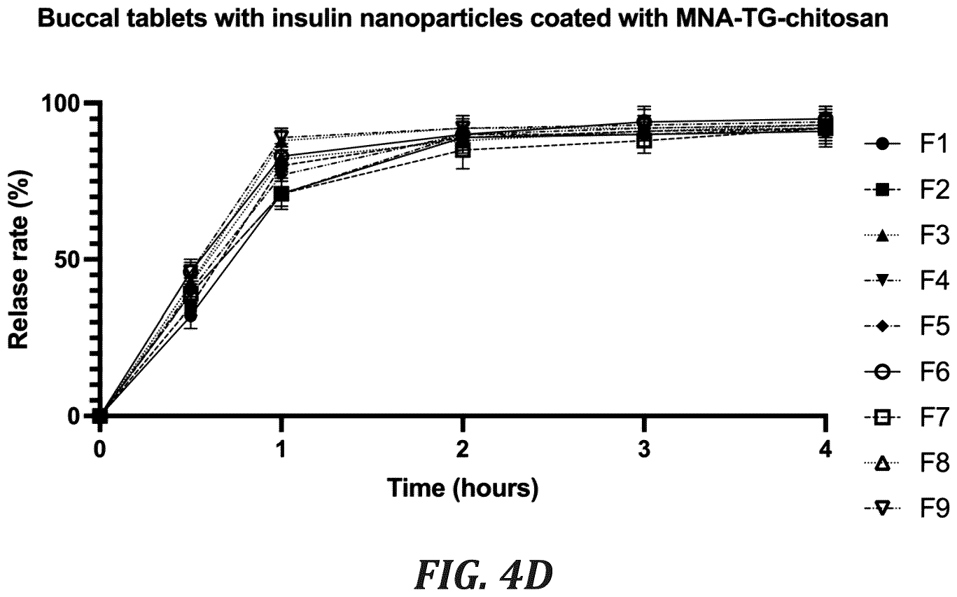

Results of ex-vitro penetration of insulin from the buccal tablets showed that tablets containing insulin particles with all different adhesive layers released almost 100% of its insulin content after 2 h, with the tablets with bioadhesive layer of F9 showed the highest penetration rate (FIG. 4). The penetration of different materials could be arranged in descending order: carbopol 934P™ >carbopol 934P™/hydroxypropyl methylcellulose >hydroxypropyl methylcellulose. Among all materials used in this case, carbopol 934P™ had a lower gel viscosity compared with hydroxypropyl methylcellulose, which it is thought to contribute to a higher mucosal penetration.

In this study, ethylcellulose generally retarded the release rate of insulin. The slow release rate might be related to the formation of a less soluble complex of ethylcellulose²⁴. It was also used as the water impermeable coating layer for the tablets. To test the uni-directional release profile of the tablets, both sides of the tablets were attached to porcine buccal tissue and tested for their release behaviors. According to FIG. 4E, less than 10% of the insulin was released within the whole 4-hour test time, which means that once the tablets are applied to patient, most of the insulin will be released from the bioadhesive layers, and very little of the insulin will leak out from the water impermeable coating layer, but this was likely due to leakage at the periphery of the tablet.

With the same bioadhesive layers, the tablets containing insulin-loaded particles (i.e. encapsulated insulin) resulted in a higher penetration rate than tablets containing free insulin (FIGS. 4A-D). MNA-TG-chitosan particles showed the highest penetration within 2 hours, followed by insulin-loaded TG-chitosan particles and insulin-loaded chitosan particles (FIGS. 4B, C, and D). It is thought that the chitosan can open the tight junctions on the epithelial cells and thus can increase the penetration of insulin through oral mucosal tissue²⁵. Additionally, TG-chitosan and MNA-TG-chitosan might have limited their complexation with insulin, since fewer chitosan amino groups are available for interaction with insulin. Subsequently, tablets containing insulin particles coated with TG-chitosan and MNA-TG-chitosan released more insulin than tablets with unmodified chitosan particles. Therefore, to maximize the penetration of insulin along with the best mucoadhesion of the tablets and to achieve uni-directional insulin release toward the buccal mucosa, the tablets with bioadhesive layer of F9 containing MNA-TG-chitosan coated insulin particles were selected for further studies.

### Example 8

### In vitro Cytotoxicity Assay

An MTT assay was employed to investigate the cytotoxicity of all insulin particles coated with chitosan, TG-chitosan, and MNA-TG-chitosan. Since the purpose of these particles was used to prepare insulin buccal tablets, all potential cells in human body that might contact the insulin particles were used in this study. Besides buccal cells (TR-146), as the liver is the primary organ where insulin performs its physiological function²⁶, liver cells (HepG2) were also used in this test. In addition, the results of the ex-vivo penetration test showed that less than 10% of the insulin could be leaked out from the edge of the tablets and might go directly into the GI tract, so intestinal cells (Caco-2) were also used in this study. All insulin particles coated with chitosan, TG-chitosan, and MNA-TG-chitosan were found to have no significant impact on the cell viability at the concentration of 50-1000 μg/ml, which indicated that all insulin particles could be safely used in the buccal tablets to reach the therapeutic window (data not shown).

### Example 9

### In vitro Cell Uptake Assay

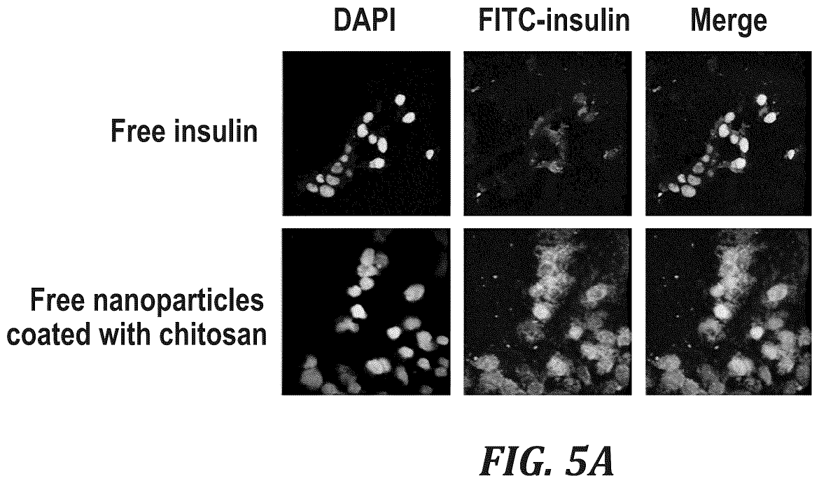

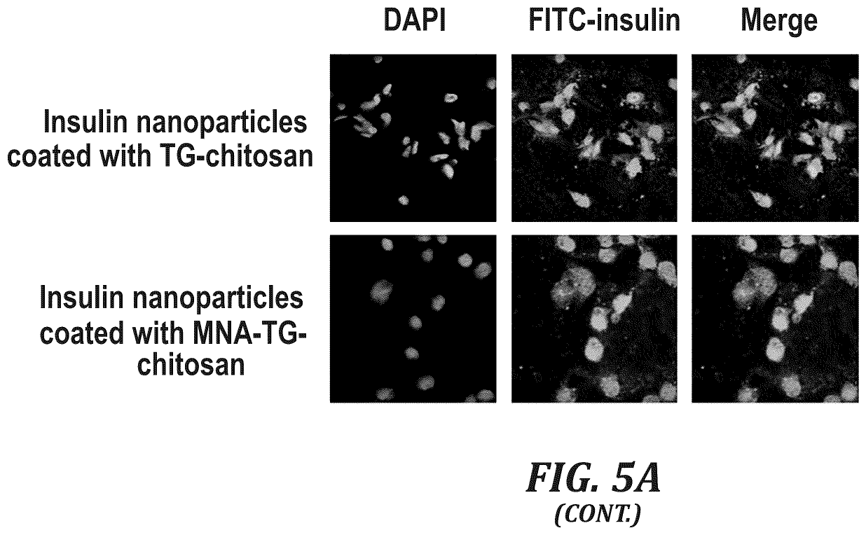

Hep G2 cell is a human liver cancer cell line commonly utilized as a hepatocyte absorption model In vitro. The cellular uptakes were quantified using flow cytometry and visually by confocal laser scanning microscopy (CLSM) observation. The intracellular fluorescence intensities (FIG. 5A) of redissolved spray-dried insulin particles coated with chitosan, TG-chitosan and MNA-TG-chitosan were 2.9, 3.6, and 4.4-fold higher than the intensity of the free FITC-insulin group, respectively (FIG. 5B). These results demonstrated that encapsulated insulin had higher cell uptake than free insulin. Besides the smaller particle sizes of particles coated with TG-chitosan and MNA-TG-chitosan, the cell uptake mechanism is also controlled by reactions between thiol functional groups on the surface of the insulin particles with exofacial thiols of transmembrane proteins²⁷. It can be done by exchanging disulfide groups on the surface of the particles with thiol groups on the transmembrane proteins so that a new disulfide bond can be formed between the particles and the membrane protein, resulting in an increased absorptive insulin²⁸. It was found that pre-activated MNA-TG-chitosan had more free —SH groups, it can bind more tightly to the cell transmembrane proteins to increase the cellular uptake.

### Example 10

### Cell Tight Junction and Insulin Transport

Chitosan-based particles are effective in enhancing drug permeation across epithelium principally by opening tight junctions that temporarily connect epithelial cells 29 temporarily.

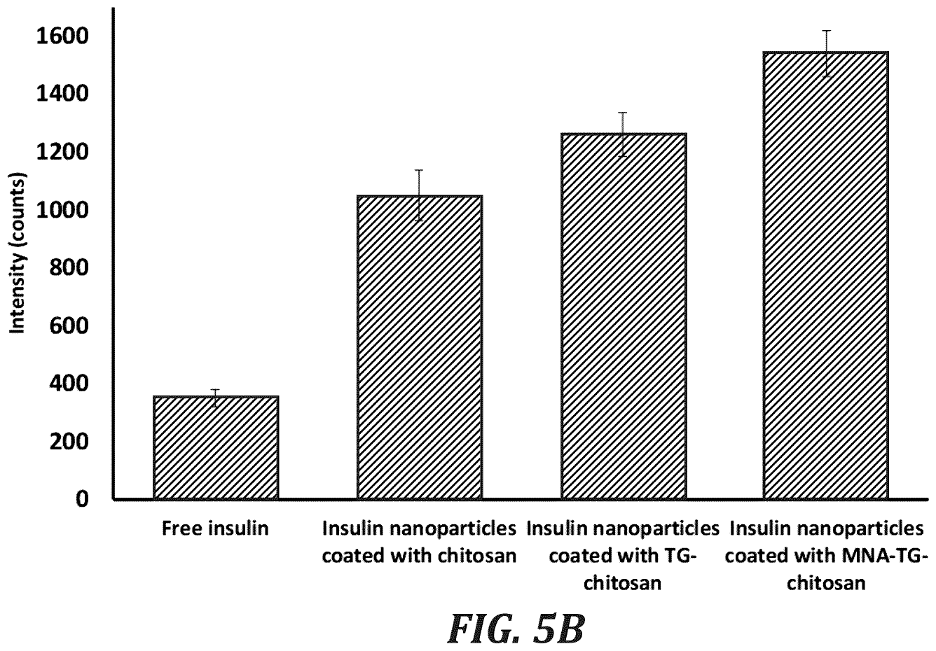

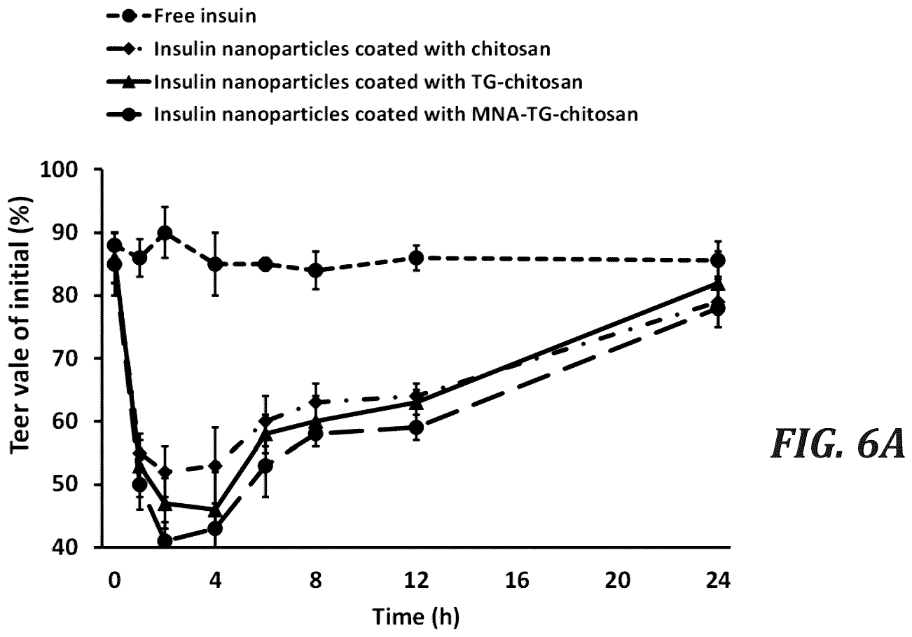

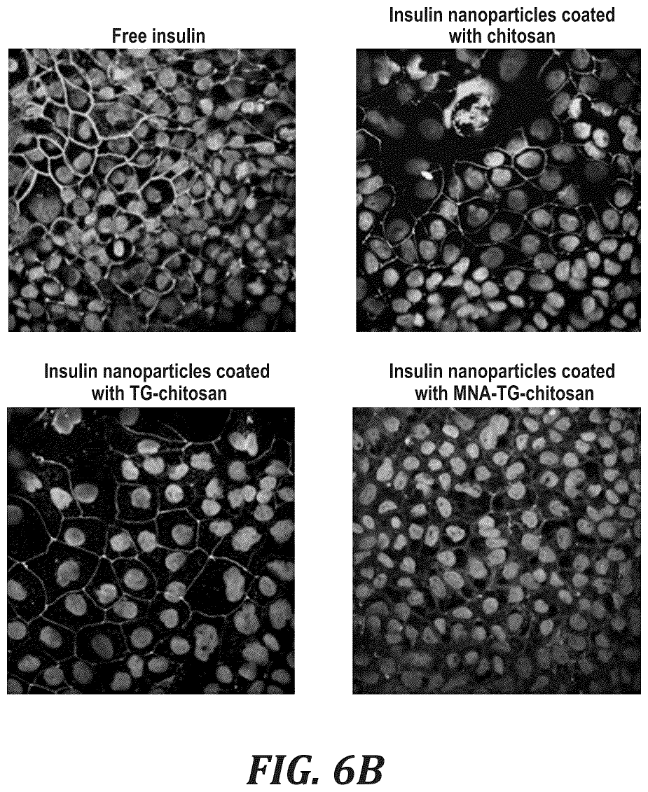

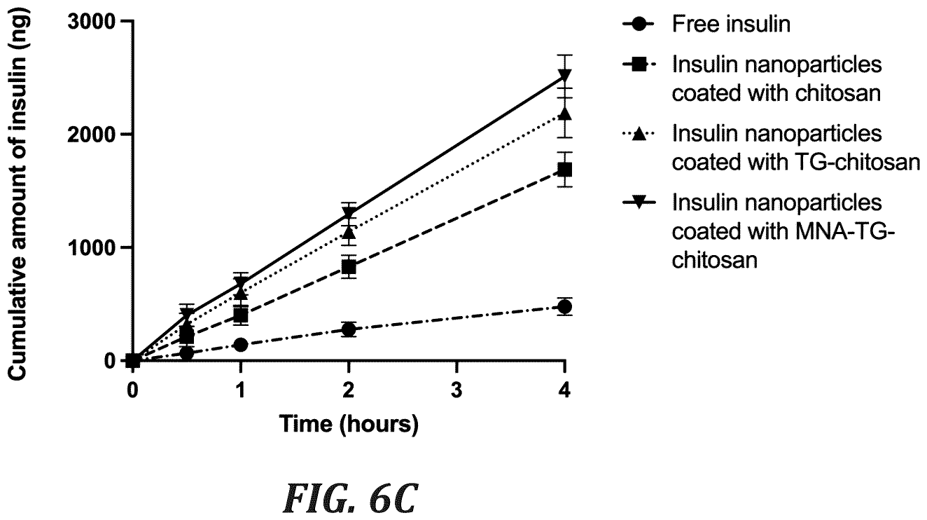

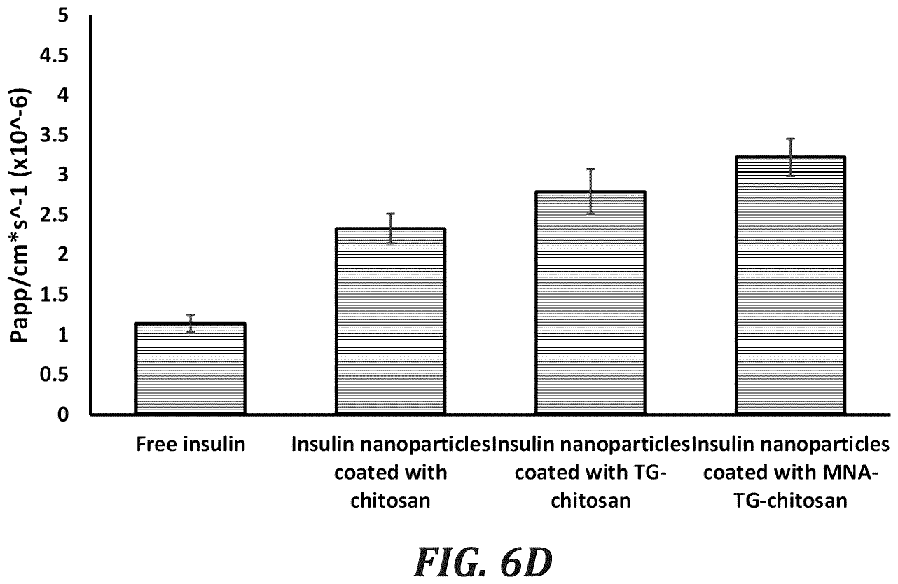

Insulin particles coated with chitosan, TG-chitosan, and MNA-TG-chitosan significantly reduced the Trans-Epithelial Electrical Resistance (TEER) value of TR146 cells compared to untreated cells and free insulin-treated cells, suggesting the opening of the tight junctions (FIG. 6A). Among these results, it can be observed that the MNA-TG-chitosan induced the highest reduction of the TEER value, followed by TG-chitosan and chitosan. Further, after the test samples were removed and the medium was refilled, a gradual recovery in TEER values was observed, suggesting that opening the tight junctions was a transient and reversible process. Moreover, as shown in FIG. 6B, the band of ZO-1 in the group treated with free insulin appeared continuously between adjacent cells. After incubation with insulin particles coated with chitosan, TG-chitosan, and MNA-TG-chitosan, the ZO-1 staining bands were segmented and discontinuous, indicating the opening of tight cell junction. Besides, the signals of ZO-1 staining were weakest for the insulin particles with MNA-TG-chitosan followed by TG-chitosan and chitosan, which was consistent with the results of TEER values. The reason TG-chitosan and MNA-TG-chitosan can open the tight junction more widely than chitosan is based on interaction with thiol groups of membrane-bound enzymes and proteins according to different mechanisms³⁰. Thiomers are capable of inhibiting protein tyrosine phosphatase through glutathione. The inhibition of this enzyme inhibits the dephosphorylation of the tyrosine subunits on occludin, thereby resulting in the opening of the tight junction³¹. In our case, as more free —SH groups were on the surface of the preactivated MNA-TG-chitosan, these particles were more capable of opening the cell tight junction compared with TG-chitosan.

The cumulative amount of insulin transported through the TR-146 monolayer was compared among the free insulin and the insulin particles coated with chitosan, TG-chitosan, and MNA-TG-chitosan (FIG. 6C). The amount of insulin transported in each group within 4 h is as follows: control group (free insulin): 368±32 ng; insulin particles coated with chitosan group: 1863±109 ng; insulin particles coated with TG-chitosan group: 2246±154 ng; insulin particles coated with MNA-TG-chitosan group: 2593±121 ng. Pₐₚₚ values or the absolute quantity of particles were more appropriate than cumulative amounts of insulin to further compare insulin transport efficiency. The apparent permeability (Pₐₚₚ) value of insulin for insulin particles coated with MNA-TG-chitosan (3.22±0.23×10⁻⁶ cm s⁻¹) was highest among all tested groups followed by TG-chitosan (2.79±0.28×10⁻⁶ cm s⁻¹), chitosan (2.33±0.19×10⁻⁶ cm s⁻¹) and free insulin (1.14±0.11×10⁻⁶ cm s⁻¹) (FIG. 6D). Accordingly, these results directly showed a higher insulin transport through the monolayer of the TR-146 cells when MNA-TG-chitosan encapsulated the insulin. Besides the fact that the TG-chitosan and MNA-TG-chitosan can open the cell tight junction, they were also proved to have efflux pump inhibitory properties. By forming disulfides with cysteine-substructures of natural proteins located within the efflux pumps channel, they can reversibly inhibit efflux pumps^{32,33} and thus increase the cell penetration.

### Example 11

### In-vivo Test—Insulin Buccal Tablets-mediated Insulin Delivery and Effect on Blood Sugar Level

All rats were given a single intravenous injection of 55 mg/kg of STZ into a tail vein under the influence of isoflurane anesthesia. Using a glucometer and glucose test strips, hyperglycemia (>16 mmol/L) was detected in tail tip blood samples for all rats after 4 days. After confirming no insulin tolerance occurred among these rats, the diabetic rat model was successfully established for further test.

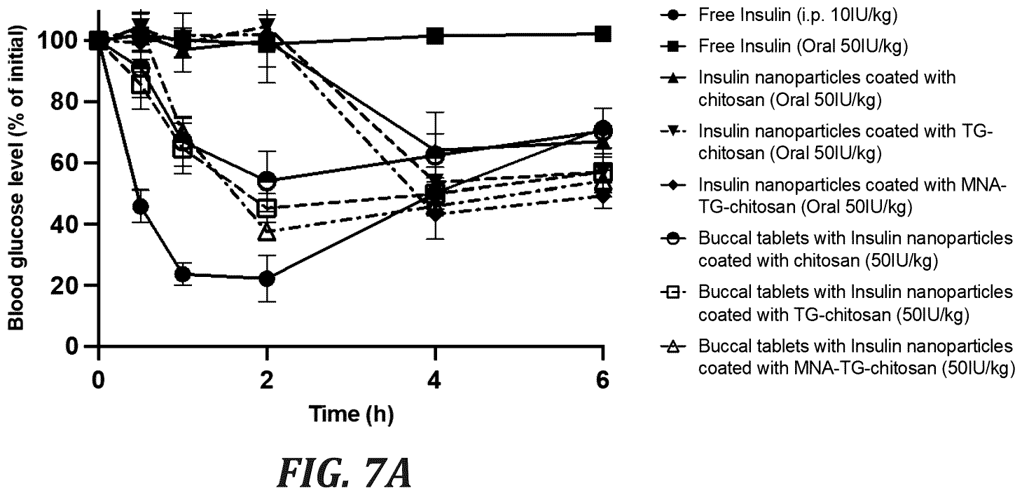

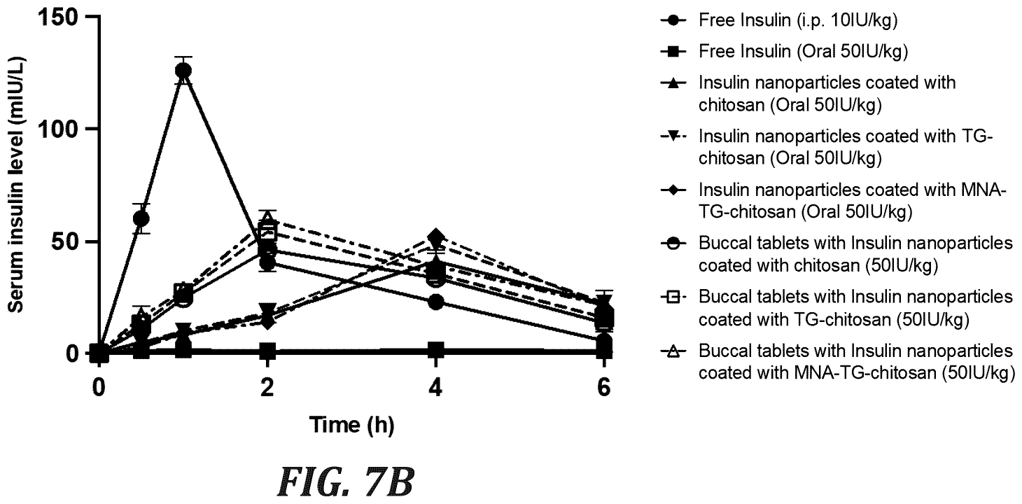

An analysis of the insulin delivery efficiency of particles and buccal tablets was conducted in established Type I diabetes rats. FIG. 7A showed an average pharmacodynamic profile for various test groups. In the positive control group that received an i.p. injection of a 10 IU/kg insulin solution, blood glucose levels dropped sharply to around 25% of the basal level within 1 hour and held at this level for the following 1 hour. Oral administration of insulin solution did not result in any significant changes in the blood glucose levels (FIG. 7A). The oral administration of insulin particles coated with chitosan, TG-chitosan, and MNA-TG-chitosan resulted in a gradual reduction in blood glucose levels by 59% and 52% and 48% of the basal level, respectively, within 4 h and held at this level for the whole testing time (FIG. 7A). However, the blood glucose reduction effect occurred after two hours, indicating particles took a considerable amount of time to reach the small intestine, where the insulin was released. Therefore, the traditional oral delivery route of insulin was a slow reaction delivery method with a longer duration. For insulin oral mucosa tablets, tablets containing particles coated with chitosan, TG-chitosan, and MNA-TG-chitosan resulted in a fast reduction in blood glucose levels by 46% and 40% and 37%, respectively, within 2 h and held at this level for the whole testing time (FIG. 7A). Notably, all rats given buccal delivered tablets were tested under anesthesia so that the buccal tablets might have a faster reaction and better blood glucose reduction effect, than if the rats were awake. Moreover, insulin particles coated with MNA-TG-chitosan and the buccal tablets containing it exhibited the highest blood glucose reduction effects, consistent with the results of in-vitro and ex-vivo tests. All the in-vivo and in-vitro results indicated that the buccal tablets containing insulin particles coated with MNA-TG-chitosan had the same fast onset of action as an injection, which can be administrated more conveniently and had a long duration of blood glucose reduction effect.

### Example 12

### Pharmacokinetics Assessment of Insulin Level in the Blood

For a deeper understanding of different dosage forms on reducing blood glucose levels, insulin levels in the blood serum were measured via ELISA. In the positive control group with i.p. injection of a 10 IU/kg insulin, blood insulin levels increased sharply to a maximal of 126.3 mIU/L within one hour, followed by a rapid decrease in the next five hours (FIG. 7B). The oral administration of insulin particles coated with chitosan, TG-chitosan, and MNA-TG-chitosan resulted in a gradual increase in insulin levels in the first four hours, with maximal levels of 41.5, 44.6 and 49.2 mIU/L, respectively, followed by a slow decreasing in next two hours (FIG. 7B). When given buccal tablets with insulin, buccal tablets containing particles coated with chitosan, TG-chitosan, and MNA-TG-chitosan resulted in a fast increase of insulin levels in the first two hours with maximal levels of 47.6, 52.0, and 55.7 mIU/L, and started slowly decreased in the following four hours (FIG. 7B). The relative insulin bioavailabilities of orally delivered insulin particles and buccal tablets were about 13% and 17% compared with the i.p. injection of insulin, indicating the buccal tablets can further optimize the bioavailability of the insulin particles. This pharmacokinetic profile further proved that the buccal tablets containing insulin particles could have similar onset of action as an injection while providing a long duration of blood glucose reduction effect. Moreover, it also can be concluded that the buccal tablets containing insulin particles coated with MNA-TG-chitosan had the highest bioavailability compared with other formulations used in this test. Accordingly, the buccal tablets containing insulin particles coated with MNA-TG-chitosan was the best formulation used in this study as it mostly increased the bioavailability of insulin.

### Example 13

### Biodistribution of Orally Delivered Insulin Particles and Insulin Buccal Tablets

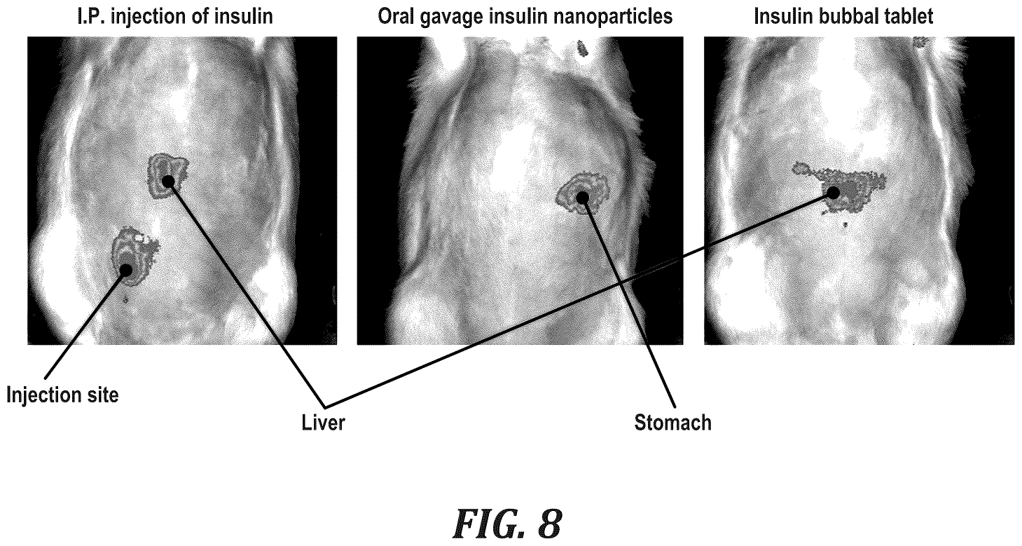

To assess whether the buccal delivery can bypass the GI tract and mimic the delivery of the i.p. injection, the biodistribution was tested in the i.p. injection of Cy5-labeled insulin, oral gavage of Cy5-labeled insulin particles, and buccal tablets containing Cy5-labeled insulin particles. As expected, almost all insulin of the rat which received i.p. injection was found in the liver³⁴ (FIG. 8), while some of the insulin remained on the injection site. In contrast, the insulin distribution of the rats who got oral gavaged insulin particles was mainly in the stomach. For the buccal tablet, it was obvious that the insulin's biodistribution was similar to the one that got the i.p. injection, in which the insulin was found in the liver. This result indicated that the insulin buccal tablets bypassed the GI tract and showed a similar delivery pattern to the i.p. injection.

### Example 14

### Toxicity Evaluation Following Oral Administration

The potential toxicity of buccal tablets containing insulin particles coated with chitosan, TG-chitosan and MNA-TG-chitosan given orally was evaluated by examining liver enzyme activities (FIG. 9)³⁵. Based on the results shown in the biodistribution test, most of the insulin released from the buccal tablets was accumulated in the liver. No significant difference was observed between groups treated with buccal tablets containing insulin particles coated with chitosan, TG-chitosan and MNA-TG-chitosan, and control group in terms of alkaline phosphatase (ALP), aspartate transaminase (AST), and alanine amino-transferase (ALT) activities. In conclusion, no toxicity was associated with the administration of buccal tablets containing insulin particles.

### Example 15

### Mucoadhesive Tablet Dosage Form Structures

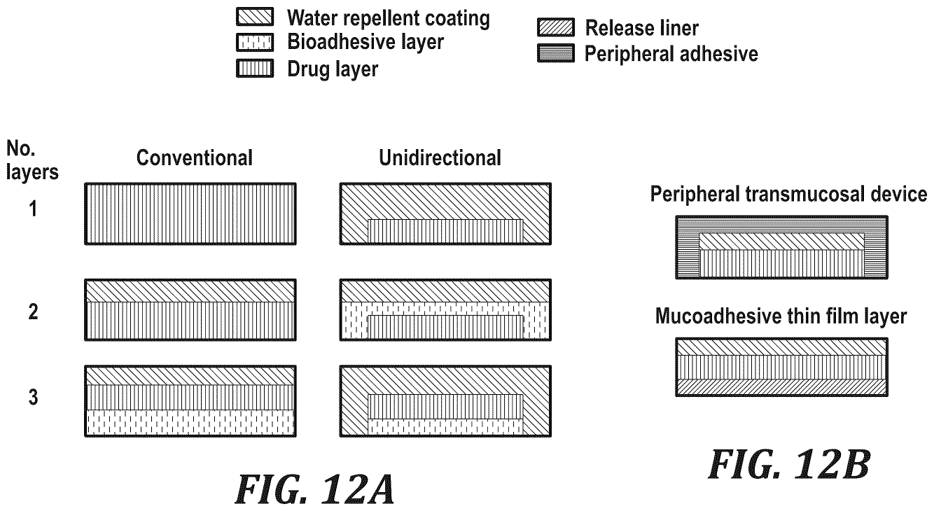

In general, pharmaceutical dosage forms (for example, tablets or films) may be composed of different layers to facilitate mucosal adhesion and to reduce the peptide dissolution into the saliva. Accordingly, pharmaceutical dosage form may contain a peptide loading layer, a 30 mucoadhesive layer, and a water impermeable layer consisting of a water-repellant polymer or coating. The combination of these three layers in different forms can allow the pharmaceutical dosage form to have a prolonged residence time at the oral mucosal surface. FIG. 12 shows multilayered dosage forma having improved unidirectional release to enhance absorption of the peptide or peptides being delivered. Using mucoadhesive polymers, tablets or films can remain in contact with the oral mucosa for minutes to a few hours. Water-insoluble polymers can be used as the water impermeable layer to create unidirectional release of the peptide to the mucosal surface and to prevent release of peptide into the saliva. A tablet design where the water impermeable layer surrounds both the peptide loading layer and the sides of the mucoadhesive layer is preferred since that is likely to reduce elution via the sides of the tablet (see middle column, bottom in FIG. 12), even though it is shown herein that less than 10% elutes in this manner. Furthermore, FIG. 12 also shows a mucoadhesive surface (*) without the water impermeable layer, to facilitate muco-adhesion of the pharmaceutical dosage form to a target mucosal tissue.

### CONCLUSION

This study presented triple-layer oral mucoadhesive tablets containing insulin particles. In order to make it more suitable for oral mucosa delivery, MNA-TG-chitosan was synthesized and was found to increase the mucoadhesive properties of the insulin particles, while maintaining small particle size and high entrapment efficiency. A particular peptide encapsulation was tested, including chitosan/sodium tripolyphosphate/insulin cross-linked particles. These particles were optimized to 318 nm of particle size, 0.18 of PDI, 99.4% of entrapment efficiency, and 25.01% of loading content. The insulin peptide particles coated with MNA-TG-chitosan exhibited the highest cell uptake and penetration efficiency compared to insulin particles coated with chitosan and TG-chitosan. In order to optimize the release profile of insulin along with the mucoadhesion of the tablets, while at the same time achieving uni-directional drug release toward the oral mucosa, triple-layer tablets were prepared. A number of factors were considered when evaluating the tablets, such as the content uniformity, weight variation, thickness, diameter, hardness, friability, swelling index, surface pH, and mucoadhesion strength. The optimized mucoadhesion force of 1.12±0.16 N was achieved by optimizing the materials used in the mucoadhesive layer. Tablets with the optimized mucoadhesive layer having insulin particles coated with MNA-TG-chitosan also exhibited the highest insulin penetration compared with tablets containing the insulin particles coated with pure chitosan and TG-chitosan. In vivo studies showed that the insulin peptide tablets can significantly decrease the blood glucose level compared with the oral administration of free insulin. It was also noted that compared with the oral administration of insulin particles, these insulin peptide tablets had a faster onset of hypoglycemic effect. The glucose level of the rats started decreasing 30 min after administration of the mucoadhesive insulin peptide tablets, while the oral administration of insulin particles started reducing the blood glucose level almost after 2 hours. These results indicated that the buccal tablets containing insulin particles had a similar fast onset of action as i.p. injection, but allow for more convenient administration and showed more sustained blood glucose reductions.

Having separate drug and mucosal layers is significant, since inclusion of the therapeutic peptide in the mucoadhesive layer would result in faster release, resulting in a flooding of the system with peptide and loss of the prolonged therapeutic effect. A slightly delayed release is useful because this ensures that the release of insulin to tissues occurs when the mucoadhesive layer interacts with the oral mucosa and opens the cell tight junctions to facilitate peptide absorption by the oral mucosa. Where the cell tight junctions are not open, any insulin released is at greater risk of loss in the saliva. Encapsulated peptides can have a mean diameter between 50-10,000 nm, but are preferably 150-400 nm.

The disclosure may be further understood by the non-limiting examples. Although the description herein contains many specific examples, these should not be construed as limiting the scope of the disclosure but as merely providing illustrations of some of the embodiments of the disclosure. For example, thus the scope of the disclosure should be determined by the appended aspects and their equivalents, rather than by the examples given.

Many of the molecules disclosed herein may contain one or more ionizable groups [groups from which a proton can be removed (e.g., —COOH) or added (e.g., amines) or which can be quaternized (e.g., amines)]. Where appropriate all possible ionic forms of such molecules and salts thereof are intended to be included individually in the disclosure herein. With regard to salts of the compounds herein, one of ordinary skill in the art can select from among a wide variety of available counter-ions those that are appropriate for preparation of salts of this disclosure for a given application. In specific applications, the selection of a given anion or cation for preparation of a salt may result in increased or decreased solubility of that salt. Every formulation or combination of components described or exemplified herein may be used to practice the disclosure, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings. Unless otherwise explained, all technical and scientific terms used herein have the same meaning as commonly understood by one of ordinary skill in the art to which a disclosed disclosure belongs.

### REFERENCES

1. Atkinson, M. A., Eisenbarth, G. S. & Michels, A. W. Type 1 diabetes. The Lancet 383, 69-82 (2014).

2. Chatterjee, S., Khunti, K. & Davies, M. J. Type 2 diabetes. The Lancet 389, 2239-2251 (2017).

3. Brod, M., Kongs0, J. H., Lessard, S. & Christensen, T. L. Psychological insulin resistance: patient beliefs and implications for diabetes management. Quality of Life Research 18, 23-32 (2009).

4. Singh, A. P., Guo, Y., Singh, A., Xie, W. & Jiang, P. Developments in encapsulation of insulin: Is oral delivery now possible? Journal of Pharmaceutical and Biopharmaceutical Research 1, 74-92 (2019).

5. Wong, C. Y., Al-Salami, H. & Dass, C. R. Recent advancements in oral administration of insulin-loaded liposomal drug delivery systems for diabetes mellitus. International Journal of Pharmaceutics 549, 201-217 (2018).

6. Fonte, P., Aratljo, F., Reis, S. & Sarmento, B. Oral insulin delivery: how far are we? Journal of Diabetes Science and Technology 7, 520-531 (2013).

7. Brake, K., Gumireddy, A., Tiwari, A., Chauhan, H. & Kumari, D. In vivo Studies for Drug Development via Oral Delivery: Challenges, Animal Models and Techniques. Pharm Anal Acta 8: 560. doi: 10.4172/2153-2435.1000560 Volume 8. Issue 8. 1000560 Pharm Anal Acta, an open access journal ISSN: 2153-2435. Vitro Research (2017).

8. Kumria, R. & Goomber, G. Emerging trends in insulin delivery: Buccal route. J Diabetol 2, 1-9 (2011).

9. Henkin, R. Inhaled insulin—intrapulmonary, intranasal, and other routes of administration: mechanisms of action. Nutrition 26, 33-39 (2010).

10. Guo, Y. et al. Production of high loading insulin nanoparticles suitable for oral delivery by spray drying and freeze drying techniques. Scientific Reports 12, 1-11 (2022).

11. Seyam, S., Nordin, N. A. & Alfatama, M. Recent progress of chitosan and chitosan derivatives-based nanoparticles: pharmaceutical perspectives of oral insulin delivery. Pharmaceuticals 13, 307 (2020).

12. Mortazavian, E., Dorkoosh, F. A. & Rafiee-Tehrani, M. Design, characterization and ex vivo evaluation of chitosan film integrating of insulin nanoparticles composed of thiolated chitosan derivative for buccal delivery of insulin. Drug Development and Industrial Pharmacy 40, 691-698 (2014).

13. Vaidya, A. & Mitragotri, S. Ionic liquid-mediated delivery of insulin to buccal mucosa. Journal of Controlled Release 327, 26-34 (2020).

14. Ayensu, I., Mitchell, J. C. & Boateng, J. S. In vitro characterisation of chitosan based xerogels for potential buccal delivery of proteins. Carbohydrate Polymers 89, 935-941 (2012).

15. Laffleur, F., Bacher, L., Vanicek, S., Menzel, C. & Muhammad, I. Next generation of buccadhesive excipient: Preactivated carboxymethyl cellulose. International Journal of Pharmaceutics 500, 120-127 (2016).

16. Maria, S. et al. Synthesis and characterization of pre-activated thiolated chitosan nanoparticles for oral delivery of octreotide. Journal of Drug Delivery Science and Technology 58, 101807 (2020).

17. Samprasit, W., Opanasopit, P. & Chamsai, B. Mucoadhesive chitosan and thiolated chitosan nanoparticles containing alpha mangostin for possible Colon-targeted delivery. Pharmaceutical Development and Technology 26, 362-372 (2021).

18. Broitman, E. Indentation hardness measurements at macro-, micro-, and nanoscale: a critical overview. Tribology Letters 65, 1-18 (2017).

19. Krampe, R. et al. Oromucosal film preparations: points to consider for patient centricity and manufacturing processes. Expert Opinion on Drug Delivery 13, 493-506 (2016).

20. Shirvan, A. R., Bashari, A. & Hemmatinejad, N. New insight into the fabrication of smart mucoadhesive buccal patches as a novel controlled-drug delivery system. European Polymer Journal 119, 541-550 (2019).

21. Roldo, M., Hornof, M., Caliceti, P. & Bernkop-Schniirch, A. Mucoadhesive thiolated chitosans as platforms for oral controlled drug delivery: synthesis and in vitro evaluation. European Journal of Pharmaceutics and Biopharmaceutics 57, 115-121 (2004).

22. Kassem, M. A., ElMeshad, A. N. & Fares, A. R. Enhanced bioavailability of buspirone hydrochloride via cup and core buccal tablets: formulation and in vitro/in vivo evaluation. International Journal of Pharmaceutics 463, 68-80 (2014).

23. Mortazavi, S. An in vitro assessment of mucus/mucoadhesive interactions. International Journal of Pharmaceutics 124, 173-182 (1995).

24. Ni, B., Liu, M. & Lu, S. Multifunctional slow-release urea fertilizer from ethylcellulose and superabsorbent coated formulations. Chemical Engineering Journal 155, 892-898 (2009).

25. Yeh, T.-H. et al. Mechanism and consequence of chitosan-mediated reversible epithelial tight junction opening. Biomaterials 32, 6164-6173 (2011).

26. Perseghin, G. Viewpoints on the way to a consensus session: where does insulin resistance start? The liver. Diabetes Care 32, S164-S167 (2009).

27. Laurent, Q. et aL Thiol-mediated uptake. Jacs Au 1, 710-728 (2021).

28. Gao, W., Li, T., Wang, J., Zhao, Y. &Wu, C. Thioether-bonded fluorescent probes for deciphering thiol-mediated exchange reactions on the cell surface. Analytical Chemistry 89, 937-944 (2017).

29. Smith, J., Wood, E. & Dornish, M. Effect of chitosan on epithelial cell tight junctions. Pharmaceutical Research 21, 43-49 (2004).

30. Albrecht, K. & Bernkop-Schniirch, A. Thiomers: forms, functions and applications to nanomedicine. Future Medicine (2007), 41-50.

31. Werle, M. & Bernkop-Schnürch, A. Thiolated chitosans: useful excipients for oral drug delivery. Journal of Pharmacy and Pharmacology 60, 273-281 (2008).

32. Grabovac, V., Laffleur, F. & Bernkop-Schnürch, A. Thiomers: Influence of molecular mass and thiol group content of poly (acrylic acid) on efflux pump inhibition. International Journal of Pharmaceutics 493, 374-379 (2015).

33. Khan, S., Faisal, S., Shams, D. F., Zia, M. & Nadhman, A. Photo-inactivation of bacteria in hospital effluent via thiolated iron-doped nanoceria. IET Nanobiotechnology 13, 875-879 (2019).

34. Sonaje, K. et al. Biodistribution, pharmacodynamics and pharmacokinetics of insulin analogues in a rat model: oral delivery using pH-responsive nanoparticles vs. subcutaneous injection. Biomaterials 31, 6849-6858 (2010).

35. Gowda, S. et al. A review on laboratory liver function tests. The Pan African Medical Journal 3 (2009).

36. Netsomboon, K., Laffleur, F. & Bernkop-Schnürch, A. P-glycoprotein inhibitors: synthesis and in vitro evaluation of a preactivated thiomer. Drug Development and Industrial Pharmacy 42, 668-675 (2016).

37. Baldelli, A., Wells, S. and Pratap-Singh, A., 2021. Impact of product formulation on spray-dried microencapsulated zinc for food fortification. Food and Bioprocess Technology, 14(12), pp.2286-2301.

38. Baldelli, A., Liang, D.Y., Guo, Y. and Pratap-Singh, A., 2023. Effect of the formulation on mucoadhesive spray-dried microparticles containing iron for food fortification. Food Hydrocolloids, 134, p.107906.

39. Baldelli, A., Cidem, A., Guo, Y., Ong, H.X., Singh, A., Traini, D. and Pratap-Singh, A., 2022. Spray freeze drying for protein encapsulation: Impact of the formulation to morphology and stability. Drying Technology, pp.1-14.

40. He, Z. et al. Scalable fabrication of size-controlled chitosan nanoparticles for oral delivery of insulin. Biomaterials 130, 28-41 (2017).

41. Boateng, J.S. et al. Functional characterisation and permeation studies of lyophilised thiolated chitosan xerogels for buccal delivery of insulin. Protein Peptide Letters (2014) 21(11):1163-75.

42. Barzoki, Z. M. et al. Optimization and development of insulin nanoparticles by new thiolated chitosan derivative with ionic gelation method using a model-based methodology. Journal of Food and Bioprocess Engineering (2019) 2(1):25-34.

43. Dünnhaupt, S. et al. In vivo evaluation of an oral drug delivery system for peptides based on S-protected thiolated chitosan. J. Conrol Release (2012) 160(3):477-85.

44. KR 100794264.

45. U.S. Pat. No. 10,639,377.

46. U.S. Pat. No. 10,130,684.

47. Ways, T. M. M. et al. Chitosan and Its Derivatives for Application in Mucoadhesive Drug

Delivery Systems. Polymers (2018) 10, 267.

48. Federer C. et al. Thiolated Chitosans: A Multi-talented Class of Polymers for Various Applications. Biomacromolecules (2021) 22:24-56.

1. A pharmaceutical dosage form, the pharmaceutical dosage form comprising:
(a) a mucoadhesive layer, the mucoadhesive layer comprising:
(i) an ethylcellulose and a polymer of acrylic acid cross-linked with allyl sucrose or allyl pentaerythritol, wherein the polymer of acrylic acid cross-linked with allyl sucrose or allyl pentaerythritol is at least 12.5 wt % of the total mucoadhesive layer; or
(ii) an ethylcellulose, a polymer of acrylic acid cross-linked with allyl sucrose or allyl pentaerythritol, wherein the polymer of acrylic acid cross-linked with allyl sucrose or allyl pentaerythritol is at least 12.5 wt % of the total mucoadhesive layer, and a hydroxypropyl methylcellulose (HPMC);
(b) a peptide loading layer, the peptide loading layer comprising an encapsulated peptide; and
(c) a water impermeable layer, selected from one or more of ethylcellulose;
polyvinylchloride; polydimethylsiloxane; hydroxypropyl methylcellulose; hemicellulose;
poly(e-caprolactone) (PCL); carboxymethyl cellulose; polyvinylacetate; propylcellulose;
polymethyl methacrylate; methacrylic acid copolymer; and cellulose acetate phthalate;
wherein the peptide loading layer resides between the mucoadhesive layer and the water impermeable layer, and wherein the water impermeable layer facilitates unidirectional movement of the encapsulated peptide through the mucoadhesive layer to a target tissue:
and wherein the encapsulated peptide is selected from one or more of the following: an insulin; an insulin derivative; an insulin analog; a pre-insulin; a pro-drug of insulin; a glucagon-like peptide 1 (GLP-1); a GLP-1 analog; a pre- GLP-1; or a pro-drug of GLP-1.

2. The pharmaceutical dosage form of claim 1, wherein the water impermeable layer forms an outer coating while leaving a mucoadhesive surface without the water impermeable layer, to facilitate muco-adhesion.

3. The pharmaceutical dosage form of claim 1, wherein the ethylcellulose in (i) or (ii) is:
at least 50 wt % of the total mucoadhesive layer; or between about 50 wt % and about 75 wt % of the total mucoadhesive layer.

4. The pharmaceutical dosage form of claim 1, wherein the polymer of acrylic acid cross-linked with allyl sucrose or allyl pentaerythritol is between about 12.5 wt % and about 50 wt % of the total mucoadhesive layer.

5. The pharmaceutical dosage form of claim 1, wherein the polymers of acrylic acid cross-linked with allyl sucrose or allyl pentaerythritol is selected from one or more of the following: Carbopol 934 NF™; Carbopol 934P NF™; Carbopol 941 NF™; Carbopol 942NF™; Carbopol 940NF™; Carbopol 974P™; Carbopol 971P™; Noveon AA-1 Polycarbophil™(formerly Carbopol 976″); Carbopol 1342™; Carbopol 1382™; Carbopol salts™; Carbopol 981 NF™; Carbopol 980 NF™; Carbopol ETD 2050™; Carbopol ETD 2020™; Carbopol ULTREZ 10™; Carbopol 1984™; Carbopol 2984™; Carbopol 5984™; and Carbopol(R) 71G NF™

6. The pharmaceutical dosage form of claim 1, wherein the polymers of acrylic acid cross-linked with allyl sucrose or allyl pentaerythritol is Carbopol 934P NF™.

7. The pharmaceutical dosage form of claim 1, wherein the mucoadhesive layer has a thickness that is at least 100 μm.

8. The pharmaceutical dosage form of claim 1, wherein the encapsulated peptide comprises: a peptide particle coated with chitosan.

9. The pharmaceutical dosage form of claim 1, wherein the encapsulated peptide comprises: a tripolyphosphate (TPP), a chitosan, and a peptide therapeutic.

10. The pharmaceutical dosage form of claim 8, wherein the chitosan is a thiolated chitosan.

11. The pharmaceutical dosage form of claim 10, wherein the thiolated chitosan is selected from: mercaptonicotinic (MNA)-thioglycolic acid-chitosan (MNA-TG-chitosan); and thioglycolytic acid-chitosan (TG-chitosan).

12. The pharmaceutical dosage form of claim 1, wherein the encapsulated peptide is a particle having a diameter between about 50 nm and about 10,000 nm.

13. The pharmaceutical dosage form of claim 1, wherein the encapsulated peptide is a particle having a diameter between about 150 nm and about 400 nm.

14. The pharmaceutical dosage form of claim 1, wherein the encapsulated peptide is only in the peptide loading layer prior to administration.

15. The pharmaceutical dosage form of, wherein the encapsulated peptide is synthesized by 1-ethyl-3-(3-dimethylaminopropyl)carbodiimide (EDC) coupling of chitosan and thioglycolytic acid (TGA), followed by thiol-disulfide exchange of the thiolated chitosan with 2,2′-disulfandiylnicotinic acid.

16. The pharmaceutical dosage form of claim 1, wherein the peptide loading layer further comprises: one or more sweetener or flavourant.

17. The pharmaceutical dosage form of claim 16, wherein the one or more sweetener or flavourant is selected from one or more of: lactose; glucose; sucrose; mannitol; xylitol; sorbitol; and trehalose.

18. The pharmaceutical dosage form of claim 16, wherein the sweetener is mannitol.

19. The pharmaceutical dosage form of claim 1, wherein the peptide loading layer further comprises a binding agent.

20. The pharmaceutical dosage form of claim 1, wherein the binding agent is sodium alginate.

21. The pharmaceutical dosage form of claim 1, wherein the insulin analog is selected from one or more of: lispro; aspart; glulisine; glargine; detemirt; detemir; degludec; neutral protamine hagedorn (NPH); and levemir.

22. The pharmaceutical dosage form of claim 1, wherein the mucoadhesive layer has a mucoadhesivity between about 1 N and about 30 N.

23. The pharmaceutical dosage form of claim 1, wherein the pharmaceutical dosage form is a tablet dosage form.

24. A method of treating diabetes, comprising administering the pharmaceutical dosage form of claim 1, to a subject in need thereof.

25. A method for oral mucosa delivery of a peptide, comprising administering the pharmaceutical dosage form of claim 1 to a subject in need thereof.

26. The method of claim 25, wherein the oral mucosa is selected from one or more of: buccal mucosa; sublingual mucosa; palate mucosa; and tongue mucosa.
